SEQUENCE LISTING (7 sequences)

SEQ ID NO: 1 - protein, 340 aa, Bifidobacterium longum
  source 1..340: note = sub_species infantis
MTTVLVTGGAGFIATHTDIELLNKGYDVISVDNYGNSSPVALERVEQITGKPVKRYDGDVRDEALMERVFTENDIDWVIHFAGLKAVGESVAKPIEYYDNNLYSTLVLLKTMRKHGVKKIIFSSSATVYGSPKELPITEETPTGGTTNPYGTSKLFQEQILRDVHVADPSWTIVLLRYFNPVGAHESGLLGEDPKGIPANLTPYVAKVAVGELKEVQVYGDDYDTPDGTGVRDYIHVVDLAKGHVAVIDHIDKEGVFVYNLGTGHGYSVLEVIKAYEKAAGHPISYTIKPRRPGDIAACYADASKAEKELGWKAELTIDDMASSSLNWQTKNPNGFRDAE

SEQ ID NO: 2 - protein, 359 aa, Bifidobacterium longum
  source 1..359: note = sub_species infantis
MNNTNEALFDVASHFALEGTVDSIEPYGDGHINTTYLVTTDGPRYILQRMNTGIFPDTVNLMRNVELVTSTLKAQGKETLDIVRTTSGDTWAEIDGGAWRVYKFIEHTMSYNLVPNPDVFREAGRAFGDFQNFLSGFDANQLTETIAHFHDTPHRFEDFKKALAADELGRAAGCGPEIEFYLSHADQYAVVMDGLRDGSIPLRVTHNDTKLNNILMDATTGKARAIIDLDTIMPGSMLFDFGDSIRFGASTALEDERDLDKVHFSTELFRAYTEGFVGELRDSITAREAELLPFSGNLLTMECGMRFLADYLEGDVYFATKYPEHNLVRSRTQIKLVREMEQRADETRAIVADVMETTK

SEQ ID NO: 3 - protein, 751 aa, Bifidobacterium longum
  source 1..751: note = sub_species infantis
MTNTGRFTLPSEENFAEKTKELAELWGADAIRNSDGTHLDEAVLALGKKIYSAYFPTRAHNEWITLHMDETPQVYLLTARILAESNAVDVPLMDGFFEEQLKPNRDADPHKYWEVVDRTTGEVVDPSGWTLDPGEDTVHVTVAVPMHEYTVSFLAYIIWDPVEMYNHLTNDWGDKEHEIPFDIYHPATRKFVFDTFEQWLKDSPQTDVVRFTTFFYQFTLLFDQKRREKVVDWFGCACTVSPRALDDFEAKYGYRLRPEDFVDGGAYNSAWRVPRKAQRDWIDFLSGFVRKNVKQLADMSHAAGKEAMMFLGDQWIGTEPYKDGFDELGLDAVVGSIGDGTTTRMIADIPGVRYTEGRFLPYFFPDTFYEGNDPSIEGLDNWRKARRAILRSPIGRMGYGGYLSLAAKFPKFVDTVTHIANEFRDIHDRTGGVAAEGELNVAILNSWGKMRSWMAFTVAHALPNKQTYSYYGILESLSGMRVNVRFISFDDVLAHGIDSDIDVIINGGPVDTAFTGGDVWTNPKLVETVRAWVRGGGAFVGVGEPSSAPRFQSGRFFQLADVLGVDEERYQTLSVDKYFPPVVPDHFITADVPVDPAAREAWEQAGYRTPLSGCGGGQSIKPLGGIDFGEPVLNTYPVNENVTLLRADGGQVQLAANDYGKGRGVYISGLPYSAANARLLERVLFYASHNEDKYAAWSSSNPECEVAHFPEQGLYCVINNTDQPQKTTVTLADGTTEDFDLPDSGIAWREA

SEQ ID NO: 4 - protein, 316 aa, Bifidobacterium longum
  source 1..316: note = sub_species infantis
MSSATAEERARRKAERKLEKQRQAAQYDLPRSMRPSVATKTITIIVLVLVLIYFLFPIYWAIIASTKTPAQMTGSNGMWFAVPLDQLPDAIATNYSKLLGWTRGNFWRWVLNSLIYSGVSALVGTIVSVMAGYATAKFNFRGKNAAIGIIMACMLMPSALLTIPMYSIFHTLHLTNTMLSIIIPCCVSPFGFFLGRTYAQSSVPDELLEAARIDGASEARIFFTIVLRLLAPAMVTIFLFLFVATWNNFLLPLMMVSSDTLKPVTLGLYGMVSLATFTDRGALMMGALLGVLPVIVLFLGLQRYWQSGLAAGAVKG

SEQ ID NO: 5 - protein, 323 aa, Bifidobacterium longum
  source 1..323: note = sub_species infantis
MTASTTHAKGKAPRRAAAKPKRSGAQKRENRTGWAFMAPFAVLFAFVFILPIIWAIYSSFFRQVTEGGGAYGGGELVNKFVGFQNFQYVITSGNFWSGVGRVLVYTLIQVPIMIIAALALAIVIDSFVVKHVTGFRLGYFLPYAIPGVVASIIWVYLYNGQISPIVKGLAAIGINVDFFSSNIVLGSMANITTWTFTGYNMLIFLAALQAIPHDLYEAARIDGANGWQIATKIKLPNVRGAALLAMLLSIVGTIQLFNEPQIMSTADPGISKAYTPMMMAMNTSQGTITPSGDGPASAIAIVMALIAGVLAVLYTLAERKVNE

SEQ ID NO: 6 - protein, 438 aa, Bifidobacterium longum
  source 1..438: note = sub_species infantis
MVSHNKRIVAAFAAVAAMGMGLAGCGGSDTAGDTKSGSDGGVVNITYMHRLPDSKGMTLVNDIVAKWNKEHPNIQVKATKFDGNASEMIKKLETDVKAGNAPDLAQVGYAEVPEVFTKGLLQDVTDEAAKYKDDFASAPFALMQVDGKTYGLPQDTGPLAYFYNAAEFEKLGITVPKTADELIETAKKTAAQGKYIMTFQPDEAMMMMSGQAGASGPWYKVDGNSWVVNTQTKGSKAVADVYQQLIDNKAALTNPRWDASFDNSIQSGQLIGTVAAAWEAPLFIDSAGGTGAGEWKVTQLGDWFGNGTKTGSDGGSGVAVLKGSKHPAEAMEFLDWFNTQVDDLVSQGLVVAATTADAKTPQKWSDYFSGQDVMAEFKTANDNMGDFTYMPGFSAVGAAMKQTAAKAADGSAKVSDVFDTAQKTSVDTLKNLGLSVKE

SEQ ID NO: 7 - protein, 416 aa, Bifidobacterium longum
  source 1..416: note = sub_species infantis
MADFANYTPGEYAKEHIRITPTTLADGRDFFYLDDDPEFVSGAKTRELKDPRPLDYRFAPHLDADGNEVPYAAPQMRRDPLTGDWIPMATARMNRPITAGPGATAKGNPLAARKPGDPYQDGEVPDTDYNVVVFENRFPSMVRVPGVSEDVTYVDGNPLWEKKLAAGRCEVICFDPNEEGLPADLPVSRLRTVVEAWAFRTAEISKMEGIEQIFPFENHGQEIGVSLAHPHGQVYCYPFIAPKMEKELQHTEAYHEKTGGNLLKDIMNSELEAGERIVMRNHSWVAYVPAAARWPLEVHVAPVRDVLTLDQLNDEERWDLASMYSHLLKRGNAFFDKGDGKGMDLPYIAAWHQAPIHDARRENYRLNLQFFSFRRAANKIKYLAGSESGMAAWISDTTPELIAKRFHELGSIDIAD

(19) United States
(12) Patent Application Publication
(10) Pub. No.: US 2023/0114520 A1
(43) Pub. Date: Apr. 13, 2023

(54) ENGINEERED BIFIDOBACTERIUM STRAINS COMPRISING A TRANSGENE

(71) Applicant: Eligo Bioscience, Paris (FR)

(72) Inventor: Xavier DUPORTET, Paris (FR)

(21) Appl. No.: 17/938,509

(22) Filed: Oct. 6, 2022

Related U.S. Application Data
(60) Provisional application No. 63/253,244, filed on Oct. 7, 2021.

(51) Int. Cl. A61K 35/745; C12N 1/20; C12N 15/52; C12N 15/74; A61P 1/14

(52) U.S. Cl. CPC A61K 35/745; C12N 1/20; C12N 15/52; C12Y 403/01024; C12Y 305/04005; C12N 15/746; A61P 1/14; C12N 2800/101; C12N 2830/002

(57) ABSTRACT

The present invention concerns a method to modulate the level of or to modify a target molecule in a subject or an environment, said method comprising:
administering in said subject or providing to said environment an engineered bacterial strain comprising
   (i) a heterologous or engineered nucleic acid involved in the expression of a molecule of interest, wherein the expression of said molecule of interest modulates directly or indirectly the level of or modify the target molecule in said subject or environment and
   (ii) an autologous gene or gene set involved in the import and/or metabolism of a milk oligosaccharide; and
further administering to said subject, or providing to said environment, said milk oligosaccharide;
whereby the level of the target molecule in said subject or environment is modulated or the target molecule is modified in said subject or environment.

### CROSS-REFERENCE TO RELATED APPLICATIONS

This application claims the benefit of U.S. Provisional Application 63/253,244, filed on Oct. 7, 2021, the contents of which is incorporated herein by reference in its entirety for all purposes.

### REFERENCE TO SEQUENCE LISTING SUBMITTED ELECTRONICALLY

The contents of the electronic sequence listing (EB2021-06a-USreg-sequencelisting.xml; Size: 9,912 bytes; and Date of Creation: Oct. 6, 2022) is herein incorporated by reference in its entirety.

### FIELD OF THE INVENTION

The present invention concerns methods to modulate the level of or modify a target molecule in a subject or an environment.

### BACKGROUND

Microbiomes, in particular within subjects, are constituted of numerous bacterial species that are most of the time beneficial to the subject.

This is the case of the intestinal microbiota, which play a critical role in many instances such as the maturation and continued education of the host immune response (Fulde et al. (2014) Immunol. Rev. 260:21-34); provide protection against pathogen overgrowth (Kamada et al. (2013) Nat. Immunol 14:685-690); influence host-cell proliferation (ljssennagger et al. (2015) Proc. Natl. Acad. Sci. USA 112:10038-43) and vascularization (Reinhardt et al. (2012) Nature 483:627-631); regulate intestinal endocrine functions (Neuman et al. (2015) FEMS Miicrobiol. Rev. 39:509-521), neurologic signaling (Yano et al. (2015) Cell 161:264-76), and bone density (Cho et al. (2012) Nature 488:621-6); provide a source of energy biogenesis (Canfora et al. (2015) Nat. Rev. Endocrinol. 11:577-591) (5 to 10% of daily host energy requirements); biosynthesize vitamins (Yatsunenko et al. (2012) Nature 486:222-7), neurotransmitters (Yano et al. (2015) Cell 161:264-76), and multiple other compounds with as yet unknown targets; metabolize bile salts (Devlin et al. (2015) Nat. Chem. Biol. 11:685-690); react to or modify specific drugs; and eliminate exogenous toxins (Haiser et al. (2013) Science 341:295-8).

Similarly, human skin, lung and vagina are home to millions of bacteria that compose the skin microbiota, the lung microbiota and the vaginal microbiota. Similar to those in our gut, skin, lung and vaginal resident microorganisms have essential roles in the protection against invading pathogens (Scharschmidt et al. (2013) Drug Discov. Today Dis. Mech. 10:e83-e89), the education of our immune system (Belkaid et al. (2014) Science 346:954-959) and the breakdown of natural products (Grice (2015) Genome Res. 25:1514-1520), or even the improvement of therapies such as immunotherapies (Dai et al. (2020) Cell Commun Signal. 18(1):90).

In many cases, it can thus be very beneficial to increase the level or potency of the beneficial molecules produced by the microbiome.

However, association studies in humans and rodents have also shown disease-related dysbiosis across a wide spectrum of common chronic disorders, including atherosclerosis, metabolic disorders, asthma, and autism spectrum disorder. Some of these observations have been combined with experimental studies, prospective studies, or both to identify putative microbiota-derived molecular mediators of pathogenic mechanisms. More particularly, some commensal bacteria, in particular some commensal bacterial species or subpopulations of bacterial strains, can produce molecules that are or become harmful for a subject.

Using antibiotics to eliminate these bacteria producing harmful molecules would be either very challenging or dangerous. Indeed, commensal bacteria are strongly engrafted in the host microbiome and belong to a robust community of microorganisms. Having evolved in sometimes a symbiotic relationship, it has been demonstrated that antimicrobial treatment effects on commensal resident bacteria are often only transitory, i.e. the bacteria are repopulating their ecological niche after treatment, or even during treatment by becoming resistant to the treatment. On the other hand, large spectrum antibiotics can sometimes lead to the opposite effect, inducing dysbiosis which is damaging for the subject.

There is thus a need for specifically reducing the level of or deactivating molecules which are harmful for a subject or a subject's microbiome, or a need for augmenting the level of molecules which are beneficial for a subject or a subject's microbiome.

Over the past few years, there has been a growing interest in the use of genetically modified bacteria to deliver molecules of therapeutic interest.

While recombinant bacteria are used in industry for the production of insulin or vaccines, researchers are increasingly interested in the direct administration of these recombinant bacteria to humans for the in situ delivery of proteins of therapeutic interest.

However, most bacteria, after administration, have to compete with resident bacteria of the subject's microbiome that very often have a strong fitness advantage in their microbiome niche compared to the administered bacterial strain(s). The administered bacteria therefore are often rapidly shed and the ones that remain present in the microbiome are at a too low dose to achieve an effect on the subject or on the subject's microbiome.

One of the solutions to effectively achieve a sustainable effect on the subject or on the subject's microbiome, in particular when using bacteria which express a heterologous molecule intended to have a beneficial effect on the subject or on the subject's microbiome, is to improve or optimize the genetic circuits involved in the production of said heterologous molecule to make them more performant, resulting in higher production of the said heterologous molecule of interest. However, such a solution is not available for all produced molecules and can be detrimental for the administered bacteria by significantly increasing the metabolic burden associated with the production of the molecule, therefore reducing its fitness compared to the resident bacteria, further reducing its colonization potential in the subject's microbiome. It is therefore still challenging to reach the required level of the heterologous molecule for it to have a beneficial effect in a subject.

There is thus still a need for a solution that can enable bacteria engineered to express heterologous molecules intended to have a beneficial effect in a subject or a subject's microbiome, to achieve such a real and durable effect.

The present invention meets this need.

The present invention relies on the capacity of specific bacterial strains to advantageously import and metabolize specific sugars, contrary to most strains of a subject's microbiome, which give them the possibility to stably colonize the subject's microbiome, when said specific sugars are provided to said subject, at levels unreachable without this competitive advantage.

Recent studies showed that it was possible to engraft a wild-type bacterial strain (also called probiotic) of Bifidobacterium longum subspecies infantis (B. infantis) into healthy adult microbiomes at a relative abundance of up to 25% of the bacterial population by co-administering human milk oligosaccharides (HMOs), which are specifically imported and metabolized by said probiotic strain (Button et al. (2022) Cell Host & Microbe 30:712-725). However, this study does not consider or suggest the interest of such a possible engraftment to produce and deliver in situ a beneficial molecule at a therapeutically efficient level.

The present invention arises from the unexpected finding by the inventors that it is possible to produce a beneficial molecule at a therapeutically efficient level in a subject, by administering to the subject a probiotic bacterial strain which is able to import and metabolize a milk oligosaccharide, and which is further engineered to produce said beneficial molecule, and further administering to said subject said milk oligosaccharide.

Because these engineered bacterial strains are able to import and metabolize milk oligosaccharide very efficiently, contrary to most other commensal strains of the treated subject's microbiome, they can reach a colonization level sufficient for producing the beneficial molecule at an overall level which is high enough to lead to a real positive effect in the subject. Moreover, the use of bacterial strains or probiotics as live vectors to deliver biologically active molecules further presents the advantage of combining: (i) the intrinsic beneficial properties of some strains and (ii) the ability to have both a steady and a local production of the protein of interest.

### SUMMARY OF THE INVENTION

Therefore, the present invention concerns a method to modulate the level of or to modify a target molecule in a subject or an environment, said method comprising:
administering in said subject or providing to said environment an engineered bacterial strain comprising
   (i) a heterologous or engineered nucleic acid involved in the expression of a molecule of interest, wherein the expression of said molecule of interest modulates directly or indirectly the level of or modify the target molecule in said subject or environment, and
   (ii) an autologous gene or gene set involved in the import and/or metabolism of a milk oligosaccharide; and
further administering to said subject, or providing to said environment, said milk oligosaccharide;
whereby the level of the target molecule in said subject or environment is modulated or the target molecule is modified in said subject or environment.

In a particular embodiment, after administration or provision of said engineered bacterial strain and said milk oligosaccharide, said engineered bacterial strain is present in the microbiome of said subject or environment at a colonization level enabling an overall production of the molecule of interest in an amount efficient for modulating the level of or modifying the target molecule at a rate leading to an effect on said subject or said environment, or on said subject's or environment's microbiome.

In a particular embodiment, after administration or provision of said engineered bacterial strain and said milk oligosaccharide, said engineered bacterial strain is present in the microbiome of said subject or environment at a colonization level higher than the natural colonization level of a natural resident bacterial strain of the same species in the microbiome of said subject or environment, during the administration period of the milk oligosaccharide and/or of the engineered bacterial strain.

In a particular embodiment, said engineered bacterial strain becomes permanently present. In an alternative embodiment, said engineered bacterial strain becomes temporarily present.

In a particular embodiment, said engineered bacterial strain becomes present at a colonization level corresponding to at least 0.1%, 0.5%, 1%, 2%, 3%, 4% or 5% of the microbiome of the subject. In a particular embodiment, said engineered bacterial strain becomes present at a colonization level corresponding to at least 7%, 10%, 15%, 20% or 25% of the microbiome of the subject.

In a particular embodiment, said method is for reducing the level of a target molecule. In that particular embodiment, said molecule of interest is preferably involved in the degradation, modification, inactivation, adsorption, absorption, and/or transport of said target molecule. In still that particular embodiment, said method can be for preventing or treating, in said subject, a disease, disorder or condition directly or indirectly associated with said target molecule.

In an alternative embodiment, said method is for increasing the level of a target molecule. In that particular embodiment, said molecule of interest is preferably involved in the expression, secretion and/or activation of said target molecule, or said molecule of interest is said target molecule. In still that particular embodiment, said method can be for preventing or treating, in said subject, a disease, disorder or condition, a therapy of which comprises said molecule of interest.

In still an alternative embodiment, said method is for modifying a target molecule. In that particular embodiment, said molecule of interest is preferably involved in the phosphorylation, glycosylation, ubiquitination, nitrosylation, methylation, acetylation and/or lipidation of said target molecule. In still that particular embodiment, said method can be for preventing or treating, in said subject, a disease or disorder associated with said target molecule.

In a particular embodiment, said engineered bacterial strain becomes present at a colonization level enabling an overall production of the molecule of interest at a therapeutically or prophylactically efficient amount.

In a particular embodiment, said milk oligosaccharide is a human milk oligosaccharide.

In a particular embodiment, said milk oligosaccharide consists of carbohydrate polymers found in mammalian milk which are not metabolized by any combination of digestive enzymes expressed from mammalian genes.

In a particular embodiment, said milk oligosaccharide is selected from the group consisting of fucosyllactose, lacto-N-fucopentose, lactodifucotetrose, sialyllactose, disialyllactone-N-tetrose, 2′-fucosyllactose, 3′-sialyllactosamine, 3′-fucosyllactose, 3′-sialyl-3-fucosyllactose, 3′-sialyllactose, 6′-sialyllactosamine, 6′-sialyllactose, difucosyllactoase, lacto-N-fucosylpentose I, lacto-N-fucosylpentose II, lacto-N-fucosylpentose III, lacto-N-fucosylpentose V, sialyllacto-N-tetraose, their derivatives and combinations thereof.

In a particular embodiment, said milk oligosaccharide is 2′-fucosyllactose (2FL), lacto-N-triose (LNT), lacto-N-neotetraose (LNnT) or any combination thereof.

In a particular embodiment, said milk oligosaccharide is a modified, recombinant or synthetic milk oligosaccharide.

In another particular embodiment, said milk oligosaccharide is obtained from human milk.

In a particular embodiment, said engineered bacterial strain naturally comprises said at least one gene involved in the import and/or metabolism of a milk oligosaccharide. In a more particular embodiment, said engineered bacterial strain naturally comprises at least one gene involved in the transport of a milk oligosaccharide.

In a particular embodiment, said engineered bacterial strain is a Bifidobacterium strain. In an alternative embodiment, said engineered bacterial strain is a Roseburia strain. In an alternative embodiment, said engineered bacterial strain is a Eubacterium strain.

In a particular embodiment, said engineered bacterial strain is from a subspecies which is not a resident Bifidobacterium subspecies of a typical adult microbiome.

In a particular embodiment, said engineered bacterial strain is not obtained from a natural resident Bifidobacterium strain of a typical adult microbiome.

In a particular embodiment, said engineered bacterial strain is an engineered Bifidobacterium strain which comprises at least one autologous gene of the H5 gene cluster from Bifidobacterium longum subsp. infantis.

In a particular embodiment, said engineered bacterial strain is a Bifidobacterium longum subsp. infantis bacteria. In a more particular embodiment, said engineered bacterial strain is obtained from Bifidobacterium longum subsp. infantis strain DSM 20218 or from Bifidobacterium longum subsp. infantis strain DSM 20088.

In a particular embodiment, the expression of the heterologous or engineered nucleic acid is regulated by said milk oligosaccharide. In a more particular embodiment, said heterologous or engineered nucleic acid is operably linked to a promoter inducible by the presence of said milk oligosaccharide. In still a more particular embodiment, said inducible promoter is not the natural promoter of said heterologous or engineered nucleic acid.

The present invention also concerns an engineered bacterial strain comprising (i) an heterologous or engineered nucleic acid involved in the expression of a molecule of interest and (ii) an autologous gene or gene set involved in the import and/or metabolism of a milk oligosaccharide.

In a particular embodiment, said milk oligosaccharide is a human milk oligosaccharide.

In a particular embodiment, said milk oligosaccharide consists of carbohydrate polymers found in mammalian milk which are not metabolized by any combination of digestive enzymes expressed from mammalian genes.

In a particular embodiment, said milk oligosaccharide is selected from the group consisting of fucosyllactose, lacto-N-fucopentose, lactodifucotetrose, sialyllactose, disialyllactone-N-tetrose, 2′-fucosyllactose, 3′-sialyllactosamine, 3′-fucosyllactose, 3′-sialyl-3-fucosyllactose, 3′-sialyllactose, 6′-sialyllactosamine, 6′-sialyllactose, difucosyllactoase, lacto-N-fucosylpentose I, lacto-N-fucosylpentose II, lacto-N-fucosylpentose III, lacto-N-fucosylpentose V, sialyllacto-N-tetraose, their derivatives and combinations thereof.

In a particular embodiment, said milk oligosaccharide is 2′-fucosyllactose (2FL), lacto-N-triose (LNT), lacto-N-neotetraose (LNnT) or any combination thereof.

In a particular embodiment, said engineered Bifidobacterium strain naturally comprises said at least one gene involved in the import and/or metabolism of a milk oligosaccharide.

In a particular embodiment, said engineered bacterial strain is a Bifidobacterium strain. In an alternative embodiment, said engineered bacterial strain is a Roseburia strain. In an alternative embodiment, said engineered bacterial strain is a Eubacterium strain.

In a particular embodiment, said engineered bacterial strain is from a subspecies which is not a commensal resident Bifidobacterium subspecies of a typical adult microbiome.

In a particular embodiment, said engineered bacterial strain is not obtained from a natural resident Bifidobacterium strain of a typical adult microbiome.

In a particular embodiment, said engineered bacterial strain is an engineered Bifidobacterium strain which comprises at least one autologous gene of the H5 gene cluster from Bifidobacterium longum subsp. infantis.

In a particular embodiment, said engineered bacterial strain is a Bifidobacterium longum subsp. infantis bacteria.

In a particular embodiment, said engineered bacterial strain is obtained from Bifidobacterium longum subsp. infantis strain DSM 20218 or from Bifidobacterium longum subsp. infantis strain DSM 20088.

In a particular embodiment, the expression of the heterologous or engineered nucleic acid is regulated by said milk oligosaccharide. In a more particular embodiment, said heterologous or engineered nucleic acid is operably linked to a promoter inducible by the presence of said milk oligosaccharide. In a more particular embodiment, said inducible promoter is not the natural promoter of said heterologous or engineered nucleic acid.

### DETAILED DESCRIPTION OF THE INVENTION

### Engineered Bacterial Strain

In the context of the invention, an engineered bacterial strain comprising an autologous gene or gene set involved in the import and/or metabolism of a milk oligosaccharide is used.

Gene Involved in the Import and/or Metabolism of a Milk Oligosaccharide

By “autologous gene” is meant herein a gene naturally present in said bacterial strain or related strains from the same species.

By “gene or gene set involved in the import of a milk oligosaccharide” is meant herein a gene or set of genes encoding a molecule enabling directly and/or indirectly the active transport of the milk oligosaccharide from the extracellular medium into the cytoplasm.

By “gene or gene set involved in the metabolism of a milk oligosaccharide” is meant herein a gene or set of genes encoding a molecule enabling, intracellularly and/or extracellularly after secretion in the extracellular medium, the degradation of the milk oligosaccharide.

In a particular embodiment, said engineered bacterial strain comprises at least one gene encoding a transporter of a milk oligosaccharide, in particular a transporter of a milk oligosaccharide selected from the group consisting of 2′-fucosyllactose (2FL), lacto-N-triose (LNT), lacto-N-neotetraose (LNnT) and any combination thereof.

In a particular embodiment, said engineered bacterial strain comprises at least one gene encoding an intracellular enzyme involved in the degradation of a milk oligosaccharide, in particular an intracellular enzyme involved in the degradation of a milk oligosaccharide selected from the group consisting of 2′-fucosyllactose (2FL), lacto-N-triose (LNT), lacto-N-neotetraose (LNnT) and any combination thereof.

In a particular embodiment, said engineered bacterial strain comprises at least one gene encoding a transporter of a milk oligosaccharide and at least one gene encoding an intracellular enzyme involved in the degradation of a milk oligosaccharide, wherein said milk oligosaccharide is preferably selected from the group consisting of 2′-fucosyllactose (2FL), lacto-N-triose (LNT), lacto-N-neotetraose (LNnT) and any combination thereof.

In a particular embodiment, said milk oligosaccharide can be from any mammalian milk source such as human, bovine, pig, rabbit, goat, sheep or camel milk. In a particular embodiment, said milk oligosaccharide is a mammalian milk oligosaccharide (MMO), more particularly a human milk oligosaccharide (HMO).

An “oligosaccharide,” as used herein, refers broadly to a carbohydrate having 3-20 sugar residues or degrees of polymerization from any source.

A “mammalian milk oligosaccharide” or “MMO”, as used herein, refers broadly to an oligosaccharide from mammalian milk, whether it is purified or enriched or detectable in a dairy product, as long as the oligosaccharide is not subject to metabolism by digestive enzymes expressed in the mammalian genome. Therefore, in a particular embodiment, said milk oligosaccharide consists of carbohydrate polymers found in mammalian milk which are not metabolized by any combination of digestive enzymes expressed from mammalian genes.

MMO include individual structures synthesized to produce carbohydrate structures known to be in a mammalian milk including milk from human, bovine, equine, porcine, goat, camel, water buffalo, and sheep. It refers broadly to those indigestible glycans, sometimes referred to as “dietary fiber”, or the carbohydrate polymers that are not hydrolyzed by the endogenous mammalian enzymes in the digestive tract (e.g., the small intestine) of the mammal. Mammalian milks contain a significant quantity of MMO that are not usable directly as an energy source for the milk-fed mammal but may be usable by microorganisms in the gut of that mammal.

The core structures of HMO consist of lactose at the reducing ends elongated by β-1-3-linked lacto-N-biose I (LNB, Galβ-3GlcNAc) and/or β-1-3/6-linked N-acetyllactosamine (LacNAc, Galβ1-4GlcNAc). These core structures can be further elongated with residues of galactose (Gal), N-acetylglucosamine (GlcNAc), N-acetylneuraminic acid (Neu5Ac) and decorated with fucose or sialic acid (see Ninonuevo et al. (2006) J Agric Food Chem 54:7471-7480). The combinatorial effect of elongation, fucosylation and sialylation produces a heterogenous mix of short-chain, long-chain and branched structures with more than 200 distinct HMO types identified to date (Kirmiz et al. (2018) Annu Rev Food Sci Technol 9:429). The type 1 tetrasaccharide Lacto-N-tetraose is one of the most highly abundant oligosaccharides in breast milk and together with its isomer Lacto-N-neotetraose (LNnT) and derivatives comprise up to 70% of the total amount of HMO (Ninonuevo et al. (2006) J Agric Food Chem 54:7471-7480).

MMO particularly comprises lacto-N-biose (LNB), lacto-N-triose (LNT), at least one oligosaccharide having a Type I core, at least one oligosaccharide having a Type II core, and/or combinations thereof. Type I or type II may be isomers of each other. MMO typically includes one or more of lacto-N-biose (LNB), N-acetyl lactosamine, lacto-N-triose, lacto-N-neotriose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose (3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), their derivatives, or combinations thereof. Other type II cores include but are not limited to trifucosyllacto-N-hexaose (TFLNH), LNnH, lacto-N-hexaose (LNH), lacto-N-fucopentaose III (LNFPIII), monofucosylated lacto-N-Hexose III (MFLNHIII), Monofucosylmonosialyllacto-N-hexose (MFMSLNH).

In a particular embodiment, said milk oligosaccharide is selected from the group consisting of lacto-N-biose, lacto-N-triose, N-acetyllactosamime, lacto-N-neotriose, lacto-N-tetraose, lacto-N-neotetraose, fucosyllactose, lacto-N-fucopentose, lactodifucotetrose, sialyllactose, disialyllactone-N-tetrose, 2′-fucosyllactose, 3′-sialyllactosamine, 3′-fucosyllactose, 3′-sialyl-3-fucosyllactose, 3′-sialyllactose, 6′-sialyllactosamine, 6′-sialyllactose, difucosyllactoase, lacto-N-fucosylpentose I, lacto-N-fucosylpentose II, lacto-N-fucosylpentose III, lacto-N-fucosylpentose V, sialyllacto-N-tetraose, their derivatives and combinations thereof.

In a more particular embodiment, said milk oligosaccharide is selected from the group consisting of lacto-N-biose, N-acetyllactosamine, and combinations thereof.

In another particular embodiment, said milk oligosaccharide is selected from 2′-fucosyllactose, lacto-N-triose, lacto-N-neotetrose and combinations thereof.

In a particular embodiment, said milk oligosaccharide is a modified, recombinant or synthetic milk oligosaccharide.

Modified, recombinant or synthetic milk oligosaccharides can be obtained by any method well-known from the skilled person, in particular by chemical synthesis (as disclosed for example in Bandara et al. (2020) Org Biomol Chem 18(9):1747-1753), biological synthesis or engineering or by fermentation (as disclosed for example in PCT application WO2015/197082).

It has been previously shown that the capacity of a Bifidobacterium bacterial strain to uptake and metabolize human milk oligosaccharides was linked to the presence, in its genome, of a functional H5 cluster.

Therefore, in some embodiments, said engineered bacterial strain is an engineered Bifidobacterium strain which comprises an autologous gene or gene set of the H5 gene cluster, in particular an autologous gene or gene set of a functional H5 gene cluster, more particularly from Bifidobacterium longum subsp. infantis.

As used herein, a “functional H5 gene cluster” refers to a cluster of genes in Bifidobacterium responsible for the uptake and metabolism of human milk oligosaccharides containing LNB. A functional H5 cluster typically comprises Blon_2175, Blon_2176 and Blon_2177. The H5 gene cluster typically comprises the following genes: Blon_2171 (which typically encodes a UDP-glucose 4-epimerase, typically a protein of sequence SEQ ID NO: 1), Blon_2173 (which typically encodes an aminoglycoside phosphotransferase, typically a protein of sequence SEQ ID NO: 2), Blon_2174 (which typically encodes a protein of sequence SEQ ID NO: 3), Blon_2175 (which typically encodes a binding-protein-dependent transport systems inner membrane component, typically a protein of sequence SEQ ID NO: 4), Blon_2176 (which typically encodes a binding-protein-dependent transport systems inner membrane component, typically a protein of sequence SEQ ID NO: 5), Blon_2177 (which typically encodes an extracellular solute-binding protein, family 1, typically a protein of sequence SEQ ID NO: 6), and galT (galactose-1-phosphate uridylyltransferase, typically of sequence SEQ ID NO: 7).

Therefore, in some embodiments, said engineered bacterial strain comprises at least one autologous gene selected from the group consisting of sequences encoding (i) a protein of sequence SEQ ID NO: 1 or having 80% or more sequence identity (e.g., 85% or more, 90% or more, 95% or more, 96% or more, 97% or more, 98% or more, 99% or more, 99.5% or more, 99.9% or more, or 100% sequence identity) with SEQ ID NO: 1, (ii) a protein of sequence SEQ ID NO: 2 or having 80% or more sequence identity (e.g., 85% or more, 90% or more, 95% or more, 96% or more, 97% or more, 98% or more, 99% or more, 99.5% or more, 99.9% or more, or 100% sequence identity) with SEQ ID NO: 2, (iii) a protein of sequence SEQ ID NO: 3 or having 80% or more sequence identity (e.g., 85% or more, 90% or more, 95% or more, 96% or more, 97% or more, 98% or more, 99% or more, 99.5% or more, 99.9% or more, or 100% sequence identity) with SEQ ID NO: 3, (iv) a protein of sequence SEQ ID NO: 4 or having 80% or more sequence identity (e.g., 85% or more, 90% or more, 95% or more, 96% or more, 97% or more, 98% or more, 99% or more, 99.5% or more, 99.9% or more, or 100% sequence identity) with SEQ ID NO: 4, (v) a protein of sequence SEQ ID NO: 5 or having 80% or more sequence identity (e.g., 85% or more, 90% or more, 95% or more, 96% or more, 97% or more, 98% or more, 99% or more, 99.5% or more, 99.9% or more, or 100% sequence identity) with SEQ ID NO: 5, (vi) a protein of sequence SEQ ID NO: 6 or having 80% or more sequence identity (e.g., 85% or more, 90% or more, 95% or more, 96% or more, 97% or more, 98% or more, 99% or more, 99.5% or more, 99.9% or more, or 100% sequence identity) with SEQ ID NO: 6, and (vii) a protein of sequence SEQ ID NO: 7 or having 80% or more sequence identity (e.g., 85% or more, 90% or more, 95% or more, 96% or more, 97% or more, 98% or more, 99% or more, 99.5% or more, 99.9% or more, or 100% sequence identity) with SEQ ID NO: 7.

It has also been shown that bacterial Roseburia or Eubacterium strains were able to uptake and metabolize human milk oligosaccharides, and that this capacity was linked to the presence, in their genome, of a HMO utilization loci, defined by the co-occurrence of GH136 and GH112 genes and the presence of an ABC transporter gene (Pichler et al. (2020). Nat Commun 11:3285).

Therefore, in some embodiments, said engineered bacterial strain is an engineered Roseburia strain which comprises at least one autologous gene of a Roseburia HMO utilization loci, as defined in Pichler et al. (2020). Nat Commun 11:3285, in particular an autologous GH136 gene, an autologous GH112 and/or an autologous ABC transporter gene.

In other embodiments, said engineered bacterial strain is an engineered Eubacterium strain which comprises at least one autologous gene of an Eubacterium HMO loci, as defined in Pichler et al. (2020). Nat Commun 11:3285, in particular an autologous GH136 gene, an autologous GH112 and/or an autologous ABC transporter gene.

As used herein, the percent identity is calculated in relation to polymers (e.g., polynucleotide or polypeptide) whose sequences have been aligned. The percent identity between the two sequences is a function of the number of identical positions shared by the sequences (i.e., % homology=# of identical positions/total # of positions ×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4: 11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using a BLOSUM62 matrix, a BLOSUM30 matrix or a PAM250 matrix, and a gap weight of 16, 14, 12, 10, 8, 6, or 4 and a length weight of 1, 2, 3, 4, 5, or 6. In a specific embodiment the BLOSUM30 matrix is used with gap open penalty of 12 and gap extension penalty of 4.

In a particular embodiment, said engineered bacterial strain naturally comprises said autologous gene or gene set involved in the import and/or metabolism of a milk oligosaccharide, as defined above. In a more particular embodiment, said engineered bacterial strain naturally comprises an autologous gene involved in the transport of a milk oligosaccharide, as defined above.

By “naturally comprise” is meant herein that said engineered bacterial strain has not been engineered to include said genes. In other words, said engineered bacterial strain is obtained from a strain already comprising said genes.

Bacterial strains naturally able to import and/or metabolize milk oligosaccharides, as defined above, are generally able to import and/or metabolize several different types of milk oligosaccharides, as defined above. However, in the context of the invention, it can be advantageous to only keep the specific genes involved in the import and/or metabolism of the specific milk oligosaccharide(s) administered or provided when implementing the method of the invention.

Therefore, in a particular embodiment, said engineered bacterial strain has been engineered to only comprise, among the genes involved in the import and/or metabolism of milk oligosaccharides, as defined above, the only gene(s) that are necessary to import and/or metabolize the milk oligosaccharide which is administered to the subject or provided to the environment in the method of the invention.

In some embodiments, said autologous gene or gene set involved in the import and/or metabolism of a milk oligosaccharide is under the control of a burden-sensing promoter. More particularly, when said engineered bacterial strain produces a molecule of interest, as defined below, synthesis of said molecule of interest may confer a burden and/or fitness cost on said engineered bacterial strain. In such embodiment, expression of said autologous gene or gene set involved in the import and/or metabolism of a milk oligosaccharide can be up-regulated when said burden-sensing promoter is induced by said burden and/or fitness cost relative to a basal level expression of said autologous gene or gene set when said burden-sensing promoter is not induced.

In other embodiments, said engineered bacterial strain further comprises an essential gene operably linked to a burden-sensing promoter, wherein expression of said autologous gene or gene set involved in the import and/or metabolism of a milk oligosaccharide and/or synthesis of said molecule of interest, confer a burden and/or fitness cost on said strain, and wherein expression of said essential gene is up-regulated when said burden-sensing promoter is induced by said burden and/or fitness cost relative to a basal level expression of said essential gene when said burden-sensing promoter is not induced.

Such burden-sensing promoters are typically disclosed in international application WO2021/160854.

Examples of burden-sensing promoters include a σ factor regulated promoter, such as σ32, σB and σS factor regulated promoters, a ribosomal RNA promoter, an HAC1-upregulated promoter comprising a UPR element, and a DNA-damage sensing promoter.

### Engineered Bacterial Strain

As used herein, the term “engineered” means that the bacterial cell of the invention has been modified by standard molecular biology techniques, typically to introduce the indicated heterologous nucleic acid or to modify an autologous nucleic acid, for example by transformation of the cell with a plasmid, by conjugation, by transduction of the cell with a bacteriophage, or by any suitable technique enabling introducing a nucleic acid sequence into a bacterial cell or enabling modifying a nucleic acid sequence in a bacterial cell. As will be understood by the skilled person, engineering of a bacterial strain implies a deliberate action to introduce or modify a nucleic acid sequence and does not cover introduction or modification of a nucleic acid sequence through natural evolution of the bacterial strain.

For example, the bacteria of the invention can be genetically engineered by transformation (chemical transformation or ultrasound transformation), transduction (using for example optionally engineered bacteriophages, or packaged phagemids technologies), conjugation, or electroporation.

As used herein, a “bacterial strain” refers to a genetic variant or subtype within a bacterial species. Therefore, a bacterial strain more particularly refers to a bacterium which remains genetically unchanged when grown or multiplied. The multiplicity of identical bacteria are included. A bacterial strain is typically obtained from the isolation of a clone, which can give birth to a population of cells obtained from a single bacterial cell or colony.

In a particular embodiment, said engineered bacterial strain is a Bifidobacterium strain.

Examples of Bifidobacterium species include Bifidobacterium adolescentis, Bifidobacterium animalis, Bifidobacterium angulatum, Bifidobacterium bifidum, Bifidobacterium bifidus, Bifidobacterium brevis, Bifidobacterium catenulatum, Bifidobacterium dentium, Bifidobacterium pseudocatenulatum, Bifidobacterium longum, in particular Bifidobacterium longum subspecies infantis.

In a particular embodiment, said engineered Bifidobacterium strain is from a species or subspecies which is not a resident Bifidobacterium species or subspecies of a typical adult microbiome.

By “resident species or subspecies of a typical adult microbiome” is meant herein the species or subspecies of a typical adult microbiome (for example a typical adult gut or skin microbiome) which are almost invariably present, and if altered, can be promptly restored, while “transient species or subspecies” can colonize the subject for short periods but tend to be eliminated by competition from the resident microorganisms of the subject's defense mechanisms.

By “typical adult microbiome” is meant herein a microbiome composition, in particular a qualitative microbiome composition, which is observed in more than 80% of a healthy adult population, more particularly in more than 85%, more than 90%, more than 91%, more than 92%, more than 93%, more than 94%, more than 95%, more than 96%, more than 97%, more than 98%, more than 99%, more than 99.1%, more than 99.2%, more than 99.3%, more than 99.4%, more than 99.5%, more than 99.6%, more than 99.7%, more than 99.8% or more than 99.9%, of a healthy adult population.

By “healthy adult population” is meant herein a population of adult subjects who are not under antibacterial, in particular antibiotic, treatment, who have not been under antibacterial, in particular antibiotic, treatment for the last 1 month, who is not under probiotic supplementation, who has not been under probiotic supplementation for the last 1 month, who is not under prebiotic supplementation, who has not been under prebiotic supplementation for the last 1 month, and who is not suffering from any disease or disorder, in particular any dysbiosis-associated or dysbiosis-causing disease or disorder.

In a particular embodiment, said engineered Bifidobacterium strain is not obtained from a natural resident Bifidobacterium strain of a typical adult microbiome.

By “natural resident of a typical adult microbiome” is meant herein the species, subspecies or strains of a typical adult microbiome, as defined above, which are almost invariably present, in the absence of any treatment modifying the composition of the adult microbiome.

Natural resident Bifidobacterium species or subspecies of the adult microbiome are well-known from the skilled person and include Bifidobacterium catenulatum, Bifidobacterium longum, Bifidobacterium adolescentis, Bifidobacterium angulatum, Bifidobacterium bifidum, Bifidobacterium pseudocatenulatum and Bifidobacterium dentium.

In a particular embodiment, said engineered Bifidobacterium strain is obtained from a Bifidobacterium strain present in less than 1% of typical adult microbiomes, as defined above, in particular less than 0.9%, less than 0.8%, less than 0.7%, less than 0.6%, less than 0.5%, less than 0.4%, less than 0.3%, less than 0.2% or less than 0.1% of typical adult microbiomes as defined above.

In a particular embodiment, said engineered Bifidobacterium strain is obtained from a natural resident Bifidobacterium strain of an infant microbiome.

Natural resident Bifidobacterium species, subspecies or strains of the infant microbiome are well-known from the skilled person and include Bifidobacterium breve, Bifidobacterium longum subspecies longum and Bifidobacterium longum subspecies infantis.

In a particular embodiment, said engineered Bifidobacterium strain is a Bifidobacterium longum subsp. infantis strain.

In a more particular embodiment, said engineered Bifidobacterium strain is a Bifidobacterium longum subsp. infantis strain which naturally comprises functional H5 gene cluster, as defined above.

Examples of Bifidobacterium longum subsp. infantis strain include strains DSM20218 (commercially available), DSM20088 (commercially available), DSM 15158 (disclosed in Osman et al. (2006) BMC Gastroenterol. 6:31), DSM 15159 (disclosed in Osman et al. (2006) BMC Gastroenterol. 6:31), M63 (available from Morinaga), ATCC 15697 (commercially available), UCD272 (available from Culture Systems Inc.), EVC001 (available from Evolve Biosystems and disclosed in international application WO2019/232284), CECT 7210 (available from Ordesa S.L.), BB-02 (available from Chr. Hansen), R0033 (available from Lallemand), and BT1 (available from Yonsei University).

In a more particular embodiment, said engineered Bifidobacterium strain is obtained from Bifidobacterium longum subsp. infantis strain DSM 20218 or from Bifidobacterium longum subsp. infantis strain DSM 20088.

In an alternative embodiment, said engineered bacterial strain is a Roseburia strain.

Examples of Roseburia species include Roseburia intestinalis, Roseburia hominis, Roseburia inulinivorans, Roseburia faecis and Roseburia cecicola. In a particular embodiment, said engineered bacterial strain is a Roseburia hominis strain or a Roseburia inulinivorans strain.

In a particular embodiment, said engineered Roseburia strain is a Roseburia strain, in particular a Roseburia hominis strain or a Roseburia inulinivorans strain, which naturally comprises a functional HMO utilization loci, as defined above.

In an alternative embodiment, said engineered bacterial strain is an Eubacterium strain.

Examples of Eubacterium species include Eubacterium aggregans, Eubacterium angustum, Eubacterium barkeri, Eubacterium brachy, Eubacterium budayi, Eubacterium callanderi, Eubacterium cellulosolvens, Eubacterium combesii, Eubacterium coprostanoligenes, Eubacterium dolichum, Eubacterium eligens, Eubacterium hallii, Eubacterium infirmum, Eubacterium limosum, Eubacterium minutum, Eubacterium multiforme, Eubacterium nitritogenes, Eubacterium nodatum, Eubacterium oxidoreducens, Eubacterium plexicaudatum, Eubacterium pyruvativorans, Eubacterium ramulus, Eubacterium rectale, Eubacterium ruminantium, Eubacterium saphenum, Eubacterium siraeum, Eubacterium sulci, Eubacterium tarantellae, Eubacterium tenue, Eubacterium tortuosum, Eubacterium uniforme, Eubacterium ventriosum, Eubacterium xylanophilum and Eubacterium yurii. In a particular embodiment, said engineered bacterial strain is a Eubacterium ramulus strain.

In a particular embodiment, said engineered Eubacterium strain is a Eubacterium strain, in particular a Eubacterium ramulus strain, which naturally comprises a functional HMO utilization loci, as defined above.

### Colonization Level

In a particular embodiment, administering to the subject, or providing the environment with the milk oligosaccharide, as defined above, makes said administered engineered bacterial strain be present in the microbiome of said subject or said environment at a colonization level corresponding to at least 0.5% of the microbiome, in particular of the gut microbiome, of the subject or of the microbiome of the environment, more particularly at least 0.6%, 0.7%, 0.8%, 0.9%, 1%, 1.5%, 2%, 3%, 4%, 5%, 6%, 7%, 8%, 9%, 10%, 15%, 20% or at least 25% of the microbiome, in particular of the gut microbiome, of the subject or of the microbiome of the environment.

In another particular embodiment, administering to the subject, or providing the environment, with the milk oligosaccharide, as defined above, makes said administered engineered bacterial strain be present in the microbiome of said subject or of said environment at a colonization level higher than the natural colonization level of a natural resident bacterial strain of the same species, as defined above, of the microbiome of said subject or of said environment, during the administration period of the milk oligosaccharide.

By “natural resident bacterial strain of the same species” is meant herein a natural resident bacterial strain of the same species as the strain from which the engineered bacterial strain of the invention is obtained.

In a more particular embodiment, administering to the subject, or providing the environment, with the milk oligosaccharide, as defined above, makes said administered engineered bacterial strain be present in the microbiome of said subject or of said environment at a colonization level higher than the natural colonization level of each natural resident bacterial strain of the same species, as defined above, of the microbiome of said subject or of said environment, during the administration period of the milk oligosaccharide.

In a more particular embodiment, administering to the subject, or providing the environment, with the milk oligosaccharide, as defined above, makes said administered engineered bacterial strain be present in the microbiome of said subject or of said environment at a colonization level higher than the natural colonization level of a natural resident bacterial subspecies of the same genera, as defined above, of the microbiome of said subject or of said environment, during the administration period of the milk oligosaccharide.

By “natural resident bacterial subspecies of the same genera” is meant herein a natural resident bacterial subspecies of the same genera as the strain from which the engineered bacterial strain of the invention is obtained.

In a more particular embodiment, administering to the subject, or providing the environment, with the milk oligosaccharide, as defined above, makes said administered engineered bacterial strain be present in the microbiome of said subject or of said environment at a colonization level higher than the natural colonization level of each natural resident bacterial subspecies of the same genera, as defined above, of the microbiome of said subject or of said environment, during the administration period of the milk oligosaccharide.

By “natural colonization level” is meant herein the colonization level of a resident strain observed in the microbiome of a subject or an environment, in the absence of any treatment affecting the microbiome composition.

As shown by Kato et al. (2017) Curr. Microbiol. 74:987-995, the colonization level of resident B. adolescentis in the adult gut microbiome is typically around 10⁹ cfu/g of wet feces; the colonization level of resident B. bifidum in the adult gut microbiome is typically around 10⁸ cfu/g of wet feces; the colonization level of resident B. catenulatum in the adult gut microbiome is typically around 10⁹ cfu/g of wet feces; the colonization level of resident B. dentium in the adult gut microbiome is typically around 10⁷ cfu/g of wet feces; and the colonization level of resident B. longum in the adult gut microbiome is typically around 10⁹ cfu/g of wet feces.

Therefore, in a particular embodiment, wherein said engineered bacterial strain is an engineered Bifidobacterium strain, said engineered Bifidobacterium strain becomes present at a colonization level of 10⁷ cfu/g of wet feces or more, 10⁸ cfu/g of wet feces or more, 10⁹ cfu/g of wet feces or more, or 10¹⁰ cfu/g of wet feces or more.

In the context of the invention, milk oligosaccharide is further administered to said subject or provided to the environment.

In a particular embodiment, said milk oligosaccharide is not used in its natural context. Typically, when the milk oligosaccharide is HMO as defined above, the subject is an adult. Indeed, as well-known from the skilled person, HMO are generally not eaten or drunk by adults.

In a particular embodiment, said engineered bacterial strain and said milk oligosaccharide are administered either together or separately. For example, the milk oligosaccharide may be provided as a solution and the engineered bacterial strain may be provided in dry form or as an enteric-coated tablet or capsule. Alternatively, a composition comprising both the engineered bacterial strain and the milk oligosaccharide, for example in the form of a non-aqueous liquid or gel composition or in dry form or as an enteric-coated tablet or capsule, can be administered. Alternatively, the milk oligosaccharide may be provided in dry form or as an enteric-coated tablet or capsule and the engineered bacterial strain may be provided in a separate dry form or as an enteric-coated tablet or capsule.

In a particular embodiment, the milk oligosaccharide can be administered prior to the administration of the engineered bacterial strain, or the milk oligosaccharide can be administered contemporaneously with the administration of the engineered bacterial strain, and/or the milk oligosaccharide can be administered after the administration of the engineered bacterial strain.

In a particular embodiment, the milk oligosaccharide is administered contemporaneously with the administration of the engineered bacterial strain and further administered after the administration of the engineered bacterial strain.

In a particular embodiment, said engineered bacterial strain becomes present at a colonization level as defined above 24 hours after the first administration of the engineered bacterial strain and/or the first administration of the milk oligosaccharide, in particular 2 days, 3 days, 4 days, 5 days, 6 days, 7 days, 2 weeks, 3 weeks, or 1 month after the first administration of the engineered bacterial strain and/or the first administration of the milk oligosaccharide.

In a particular embodiment, said engineered bacterial strain remains present at a colonization level as defined above (not necessarily at the same level) for 24 hours after the first administration of the engineered bacterial strain and/or the first administration of the milk oligosaccharide, in particular for 2 days, 3 days, 4 days, 5 days, 6 days, 7 days, 2 weeks, 3 weeks, 1 month, 2 months, 3 months, 6 months, or 1 year after the first administration of the engineered bacterial strain and/or the first administration of the milk oligosaccharide.

In a particular embodiment, said colonization level is maintained (not necessarily at the same level) for the whole period during which the engineered bacterial strain and/or the milk oligosaccharide is administered, in particular is regularly administered.

In a particular embodiment, said engineered bacterial strain becomes permanently present.

By “permanently present” is meant herein that said engineered bacterial strain remains present in the microbiome of the subject at a detectable level even after stopping administering milk oligosaccharide and/or said engineered bacterial strain to said subject, in particular at least one month, more particularly at least 3 months, at least 6 months or at least 1 year after after stopping administering milk oligosaccharide and/or said engineered bacterial strain to said subject.

In an alternative embodiment, said engineered bacterial strain becomes temporarily present.

By “temporarily present” is meant herein that said engineered bacterial strain is not present in the microbiome of the subject at a detectable level 1 week after stopping administering milk oligosaccharide and/or said engineered bacterial strain to said subject, more particularly 5 days, 4 days, 3 days, 2 days, or one day after stopping administering milk oligosaccharide and/or said engineered bacterial strain to said subject.

In a particular embodiment, said engineered bacterial strain does not comprise any antibiotic-resistance gene or marker.

In a particular embodiment, said engineered bacterial strain is auxotrophic. In a more particular embodiment, said engineered bacterial strain comprises an auxotrophic selection marker such as alr (alanine racemase), thyA (Thymidylate synthase), dapA (4-hydroxy-tetrahydrodipicolinate synthase). In a more particular embodiment, said engineered bacterial strain is auxotrophic to the nutrient source as defined above.

In a particular embodiment, said engineered bacterial strain further comprises a nucleic acid, in particular a heterologous or engineered nucleic acid, involved in the expression of a molecule of interest, in particular a molecule of interest having a beneficial effect for the subject or environment, or for the subject's or environment's microbiome.

In a particular embodiment, the method of the invention further includes administering to the subject or providing to the environment a prebiotic.

Prebiotics include, but are not limited to, amino acids, biotin, fructo-oligosaccharide, galacto-oligosaccharides, hemicelluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carrageenan), oligofructose, oligodextrose, tagatose, resistant maltodextrins (e.g., resistant starch), trans-galactooligosaccharide, pectins (e.g., homogalacturonan, citrus pectin, apple pectin, and rhamnogalacturonan-1), dietary fibers (e.g., soy fiber, sugarbeet fiber, pea fiber, corn bran, and oat fiber) and xylooligosaccharides.

In a particular embodiment, said prebiotic is not the milk oligosaccharide as defined above.

### Modulation of the Level or Modification of a Target Molecule Through Expression of a Molecule of Interest Encoded by a Heterologous or Engineered Nucleic Acid

In the context of the invention, the engineered bacterial strain comprises a heterologous or engineered nucleic acid involved in the expression of a molecule of interest, wherein the expression of said molecule of interest directly or indirectly modulates the level of or modifies the target molecule in said subject or environment.

### Modulation and Modification of a Target Molecule

By “molecule” is meant herein any type of molecule, such as nucleic acids, peptides, polypeptides, proteins, carbohydrates, lipids, small compounds, metabolites, organic acids, alcohols, etc. In a particular embodiment, said molecule is a peptide, polypeptide or protein.

When referring to the target molecule, the term “level” means the amount or concentration of said specific target molecule in the subject.

By “modulate” is meant herein directly or indirectly increasing or decreasing the level of the target molecule.

By “modify” is meant herein changing the chemical composition of the target molecule. Examples of modification, in particular of a protein target molecule, include phosphorylation, glycosylation, ubiquitination, nitrosylation, methylation, acetylation and/or lipidation.

In the context of the invention, said target molecule produces an effect on the subject or on the environment, or on the subject's or environment's microbiome.

In a particular embodiment, said target molecule produces a harmful effect on the subject or on the environment, or on the subject's or environment's microbiome.

In the context of the invention, by “molecule which produces a harmful effect” is meant herein (i) a molecule which is involved in the triggering, and/or the maintenance and/or the progression and/or the worsening of a disease, disorder, or unesthetic aspect in a subject, (ii) a molecule which is responsible for specific symptoms associated with a disease, disorder or unesthetic aspect in a subject, or (iii) a molecule which is responsible for a lack of efficiency of therapeutic and/or prophylactic treatments against a disease or disorder. Examples of such target molecules which produce a harmful effect are disclosed in more detail in the section “Method of preventing or treating a disease, disorder or condition” below.

In the embodiment where said target molecule produces a harmful effect, the method is preferably for reducing the level of said target molecule or for modifying said target molecule. In that particular embodiment, said method can be for preventing or treating, in said subject, a disease or a disorder associated with said target molecule.

By “reduction of the level of the target molecule” is meant herein a decrease in the level of the target molecule after the engineered bacterial strain and the milk oligosaccharide are administered compared to the level of said target molecule in the absence of any administration of said engineered bacterial strain and milk oligosaccharide.

In a particular embodiment, the reduction of the level of the target molecule is a statistically significant decrease in the level of the target molecule.

In a particular embodiment, said reduction of the level of the target molecule or said modification of the target molecule is observed 30 min after the first administration of the engineered bacterial strain and/or the first administration of the milk oligosaccharide, in particular 1 hour, 2 hours, 3 hours, 5 hours, 8 hours, 10 hours, 12 hours, 18 hours, 24 hours, 2 days, 3 days, 4 days, 5 days, 6 days, 7 days, 2 weeks, 3 weeks, or 1 month after the first administration of the engineered bacterial strain and/or the first administration of the milk oligosaccharide.

In a particular embodiment, said reduction of the level of the target molecule or said modification of the target molecule is maintained (not necessarily at the same level) for 30 min after the first administration of the engineered bacterial strain and/or the first administration of the milk oligosaccharide, in particular for 1 hour, 2 hours, 3 hours, 5 hours, 8 hours, 10 hours, 12 hours, 18 hours, 24 hours, 2 days, 3 days, 4 days, 5 days, 6 days, 7 days, 2 weeks, 3 weeks, 1 month, 2 months, 3 months, 6 months, or 1 year after the first administration of the engineered bacterial strain and/or the first administration of the milk oligosaccharide.

In a particular embodiment, said reduction of the level of the target molecule or said modification of the target molecule is maintained (not necessarily at the same level) for the whole period during which the engineered bacterial strain and/or the milk oligosaccharide is administered, in particular is regularly administered.

In the context of the invention, the reduction of the level of the target molecule or the modification of the target molecule by administration of the engineered bacterial strain is due to the fact that the engineered bacterial strain becomes present in said subject or environment at a colonization level enabling an overall production of the molecule of interest in an amount efficient for reducing the level of or modifying the target molecule at a rate leading to a beneficial effect on said subject or said environment, or on said subject's or environment's microbiome.

In an alternative embodiment, said target molecule produces a beneficial effect on a subject or environment, or on a subject's or environment's microbiome.

In the context of the invention, by “molecule which produces a beneficial effect” is meant herein (i) a molecule which is involved in the maintenance of a good health or a good esthetic aspect in a subject, (ii) a molecule which is involved in the maintenance of a good health of a microbiome, (iii) a molecule which is involved in the treatment or prevention of a disease, disorder or unesthetic aspect in a subject, (iv) a molecule which increases the efficacy of a therapy against a disease, disorder or unesthetic aspect in a subject, or (v) a molecule which prevents the decrease of efficacy of a therapy against a disease, disorder or unesthetic aspect in a subject. Examples of such target molecules which produce a beneficial effect are disclosed in more detail in the section “Method of preventing or treating a disease, disorder or condition” below.

In the embodiment where said target molecule produces a beneficial effect, the method is preferably for increasing the level of a target molecule. In that particular embodiment, said method can be for preventing or treating, in said subject, a disease or disorder, a therapy of which comprises said molecule of interest. Alternatively, in that particular embodiment, said method can be for increasing or maintaining the efficacy of a therapy against a disease or disorder in said subject, wherein the efficacy of said therapy is directly or indirectly modulated by said molecule of interest.

By “increase of the level of the target molecule” is meant herein an augmentation of the level of the target molecule after the engineered bacterial strain and the milk oligosaccharide are administered compared to the level of said target molecule in the absence of any administration of said engineered bacterial strain and milk oligosaccharide.

In a particular embodiment, the increase of the level of the target molecule is a statistically significant increase of the level of the target molecule.

In a particular embodiment, said increase of the level of the target molecule is observed 30 min after the first administration of the engineered bacterial strain and/or the first administration of the milk oligosaccharide, in particular 1 hour, 2 hours, 3 hours, 5 hours, 8 hours, 10 hours, 12 hours, 18 hours, 24 hours, 2 days, 3 days, 4 days, 5 days, 6 days, 7 days, 2 weeks, 3 weeks, or 1 month after the first administration of the engineered bacterial strain and/or the first administration of the milk oligosaccharide.

In a particular embodiment, said increase of the level of the target molecule is maintained (not necessarily at the same level) for 30 min after the first administration of the engineered bacterial strain and/or the first administration of the milk oligosaccharide, in particular for 1 hour, 2 hours, 3 hours, 5 hours, 8 hours, 10 hours, 12 hours, 18 hours, 24 hours, 2 days, 3 days, 4 days, 5 days, 6 days, 7 days, 2 weeks, 3 weeks, 1 month, 2 months, 3 months, 6 months, or 1 year after the first administration of the engineered bacterial strain and/or the first administration of the milk oligosaccharide.

In a particular embodiment, said increase of the level of the target molecule is maintained (not necessarily at the same level) for the whole period during which the engineered bacterial strain and/or the milk oligosaccharide is administered, in particular is regularly administered.

In the context of the invention, the increase of the level of the target molecule by administration of the engineered bacterial strain is due to the fact that the engineered bacterial strain becomes present in the microbiome of said subject at a colonization level enabling an overall production of the molecule of interest in an amount efficient for increasing the level of the target molecule at a rate leading to an effect on said subject or said environment, or on said subject's or environment's microbiome.

### Heterologous or Engineered Nucleic Acid Involved in the Expression of a Molecule of Interest

In the context of the invention, the modulation of the level or the modification of the molecule is mediated by the expression of a molecule of interest encoded by an heterologous or engineered nucleic acid, or which expression is regulated by an heterologous or engineered nucleic acid in said engineered bacterial strain.

By “heterologous nucleic acid” is meant herein any piece of a nucleic acid molecule (for example, DNA) which is inserted by artifice into a cell either transiently or permanently, and becomes part of the organism if integrated into the genome or maintained extrachromosomally. Such an heterologous nucleic acid can be at least a portion of an open reading frame of a gene which is partly or entirely heterologous (i.e., foreign) to the engineered bacteria, or may represent an open reading frame or a portion thereof of a gene homologous to an endogenous gene of the engineered bacteria, which portion optionally encodes a polypeptide with substantially the same activity as the corresponding full length polypeptide, e.g., wild-type polypeptide, or at least one activity of the corresponding full length polypeptide. Such an heterologous nucleic acid can alternatively be a sequence involved in the regulation of the expression of a gene such as a promoter, an operator, a terminator, a nucleic acid encoding a transcription factor, a nucleic acid encoding a repressor, a nucleic acid encoding an activator, or a nucleic acid encoding an inducer.

In a particular embodiment, said heterologous nucleic acid is from another strain than the engineered bacterial strain, more particularly from another species, still particularly from another genera, still particularly from another family, still particularly from another order, still particularly from another class, still particularly from another phylum, still particularly from another kingdom than the engineered bacterial strain.

By “engineered nucleic acid” is meant herein a nucleic acid, autologous to said bacterial strain, but which has been modified by standard molecular biology techniques, typically to introduce a mutation in the sequence of said autologous nucleic acid, in such a way that the activity of the nucleic acid or of the protein encoded by said nucleic acid is modified. As will be understood by the skilled person, engineering of a nucleic acid implies a deliberate action to introduce a modification in the nucleic acid sequence and does not cover mutation of a nucleic acid sequence through natural evolution of the bacterial strain.

Said engineered nucleic acid may be any piece of a nucleic acid molecule such as a gene or a portion thereof, a portion of an open reading frame of a gene, or a sequence involved in the regulation of the expression of a gene such as a promoter, an operator, a terminator, a nucleic acid encoding a transcription factor, a nucleic acid encoding a repressor, a nucleic acid encoding an activator, or a nucleic acid encoding an inducer.

By “molecule of interest” is meant any type of molecule, such as nucleic acids, peptides, polypeptides, proteins, carbohydrates, lipids, small compounds, metabolites, organic acids, alcohols, etc. In a particular embodiment, said molecule of interest is a protein, in particular an enzyme.

By “expression of a molecule of interest” is meant herein the direct or indirect expression of a molecule of interest. In particular, said molecule of interest can directly be expressed by said heterologous or engineered nucleic acid, and then be secreted, membrane displayed or kept intracellularly by said engineered bacterial strain. Therefore, in a particular embodiment, the molecule of interest is expressed, secreted and/or displayed by the engineered bacterial strain.

As will be understood by the skilled person, the choice of the molecule of interest the expression of which is mediated by the heterologous or engineered nucleic acid comprised by said engineered bacterial strain will depend on the target molecule the level of which is to be modulated or which is to be modified. Examples of such molecules of interest are disclosed in more detail in the section “Method of preventing or treating a disease, disorder or condition” below.

In the embodiment where said method is for reducing the level of a target molecule as defined above, said molecule of interest is preferably involved in the degradation, inactivation, adsorption, absorption, and/or transport of said target molecule.

In the embodiment where said method is for increasing the level of a target molecule as defined above, said molecule of interest is preferably involved in the expression, secretion and/or activation of said target molecule, or said molecule of interest is said target molecule or a pro-form of said target molecule.

In the embodiment where said method is for modifying a target molecule as defined above, said molecule of interest is preferably involved in the phosphorylation, glycosylation, ubiquitination, nitrosylation, methylation, acetylation and/or lipidation of said target molecule.

Without being bound by theory, the modulation or modification of the target molecule by the expressed molecule of interest is due to the fact that the engineered bacterial strain becomes present in said subject or environment at a colonization level enabling an overall production of the molecule of interest in an amount efficient for modulating the level of or modifying the target molecule at a rate leading to a beneficial effect on said subject or said environment, or on said subject's or environment's microbiome.

Therefore, in a particular embodiment, said engineered bacterial strain becomes present at a colonization level enabling an overall production of the molecule of interest at a therapeutically or prophylactically efficient amount.

Typically, said heterologous nucleic acid has been incorporated into the bacterial cell's chromosomal or extrachromosomal expression system, or as extrachromosomal expression system, by genetic engineering techniques known in the art. Typically, said engineered nucleic acid has been modified by genetic engineering techniques known in the art.

In some embodiments, said heterologous or engineered nucleic acid encoding said molecule of interest is under the control of a high expression promoter. In particular embodiments, said heterologous or engineered nucleic acid is under the control of an inducible promoter, constitutive promoter, native promoter (e.g. native to the bacterial cell), heterologous promoter, or a promoter associated with said nucleic acid in its native form.

In a particular embodiment, the expression of the heterologous or engineered nucleic acid is regulated by the presence of said milk oligosaccharide in the environment of said engineered bacterial strain. In a more particular embodiment, said heterologous or engineered nucleic acid is operably linked to a promoter inducible by the presence of said milk oligosaccharide in the environment of said engineered bacterial strain. In a particular embodiment, said heterologous or engineered nucleic acid is chromosomally integrated after a promoter that is modulated by the presence of said milk oligosaccharide in the environment of said engineered bacterial strain.

In still a more particular embodiment, said inducible promoter is not the natural promoter of said heterologous or engineered nucleic acid.

In a more particular embodiment, said heterologous nucleic acid is chromosomally integrated after an autologous promoter that is modulated by the presence of said milk oligosaccharide in the environment of said engineered bacterial strain. In such embodiment, said heterologous nucleic acid may replace the autologous gene naturally located after said autologous promoter, or may be integrated upstream or downstream the autologous gene naturally located after said autologous promoter.

In a particular embodiment, said heterologous nucleic acid is chromosomally integrated upstream of an autologous gene involved in the import and/or metabolism of said milk oligosaccharide, in particular as part of a single operon including said autologous gene involved in the import and/or metabolism of said milk oligosaccharide. Such a location of the heterologous nucleic acid is advantageous to prevent loss of function of the heterologous nucleic acid. Indeed, in case the heterologous nucleic acid is not expressed due to a frameshift mutation and/or a point mutation leading to a STOP codon occurring in the heterologous nucleic acid, the downstream autologous gene involved in the import and/or metabolism of said milk oligosaccharide will not be expressed anymore, and the engineered bacterial strain will not be able to import and/or metabolize said oligosaccharide, thereby losing its competitive advantage.

In a particular embodiment, said heterologous nucleic acid is chromosomally integrated upstream of an autologous essential gene, in particular as part of a single operon including said autologous essential gene. Such a location of the heterologous nucleic acid is advantageous to prevent loss of function of the heterologous nucleic acid. Indeed, in case the heterologous nucleic acid is not expressed due to a frameshift mutation and/or a point mutation leading to a STOP codon occurring in the heterologous nucleic acid, the downstream autologous essential gene will not be expressed anymore, and the engineered bacterial strain will die.

In a particular embodiment, said heterologous or engineered nucleic acid is a promoter inducible by the presence of said milk oligosaccharide in the environment of said engineered bacterial strain.

In a particular aspect of the invention, the bacterial strain used in the context of the invention is engineered in situ. In other words, in all the methods of the invention, the method can alternatively comprise:
administering to the subject or providing to the environment a bacterial delivery vehicle for delivery into a bacterial strain of interest comprising an autologous gene or gene set involved in the import and/or metabolism of a milk oligosaccharide, as defined above,
   wherein said bacterial delivery vehicle comprises:
      (a) a heterologous nucleic acid involved in the expression of a molecule of interest, as defined above, or
      (b) a nucleic acid encoding a gene editing enzyme/system designed to modify the genome of said bacterial strain of interest so that said bacterial strain of interest expresses a molecule of interest, as defined above,
   whereby said bacterial strain of interest is engineered in situ to express said molecule of interest, and
further administering to said subject or providing to said environment said milk oligosaccharide,
   whereby the level of the target molecule in said subject or environment is modulated or the target molecule is modified in said subject or environment.

By “bacterial delivery vehicle” is meant herein any mean that allows the transfer of a payload into a bacterium.

There are several types of delivery vehicle encompassed by the present invention including, without limitation, bacteriophage scaffold, virus scaffold, chemical based delivery vehicle (e.g., cyclodextrin, calcium phosphate, cationic polymers, cationic liposomes), protein-based or peptide-based delivery vehicle, lipid-based delivery vehicle, nanoparticle-based delivery vehicles, non-chemical-based delivery vehicles (e.g., transformation, electroporation, sonoporation, optical transfection), particle-based delivery vehicles (e.g., gene gun, magnetofection, impalefection, particle bombardment, cell-penetrating peptides) or donor bacteria (conjugation).

Any combination of delivery vehicles is also encompassed by the present invention.

The delivery vehicle can refer to a bacteriophage derived scaffold and can be obtained from a natural, evolved or engineered capsid.

In some embodiments, the delivery vehicle is the payload as bacteria are naturally competent to take up a payload from the environment on their own.

In a particular embodiment, said bacterial delivery vehicle is a packaged phagemid, said included genes and nucleic acids being located on the phagemid.

### Method of Preventing or Treating a Disease, Disorder or Condition

The present invention also concerns a method for preventing and/or treating, in a subject, a disease, disorder or condition directly or indirectly associated with a target molecule, said method comprising administering to the subject a therapeutically efficient amount of (a) an engineered bacterial strain, as defined above and (b) a milk oligosaccharide,
wherein said engineered bacterial strain comprises:
   (i) a heterologous or engineered nucleic acid involved in the expression of a molecule of interest, wherein the expression of said molecule of interest directly or indirectly decreases the level of or modifies the target molecule in said subject, and
   (ii) an autologous gene or gene set involved in the import and/or metabolism of a milk oligosaccharide,
whereby the disease, disorder or condition directly or indirectly associated with the target molecule is prevented or treated.

The present invention also concerns a composition or combination comprising an engineered bacterial strain and milk oligosaccharide for use in a method for preventing and/or treating, in a subject, a disease, disorder or condition directly or indirectly associated with a target molecule, wherein said engineered bacterial strain comprises:
(i) a heterologous or engineered nucleic acid involved in the expression of a molecule of interest, wherein the expression of said molecule of interest directly or indirectly decreases the level of or modifies the target molecule in said subject, and
(ii) an autologous gene or gene set involved in the import and/or metabolism of a milk oligosaccharide.

The present invention also concerns the use of an engineered bacterial strain and a milk oligosaccharide for the manufacture of a medicament or a pharmaceutical combination intended for the prevention and/or the treatment, in a subject, of a disease, disorder or condition directly or indirectly associated with a target molecule, wherein said engineered bacterial strain comprises:
(i) an heterologous or engineered nucleic acid involved in the expression of a molecule of interest, wherein the expression of said molecule of interest directly or indirectly decreases the level of or modifies the target molecule in said subject, and
(ii) an autologous gene or gene set involved in the import and/or metabolism of a milk oligosaccharide.

In the context of the invention, a “disease, disorder or condition associated with a target molecule” is a disease, disorder or condition, the onset and/or maintenance of which is due directly or indirectly to the target molecule.

In a particular embodiment, the target molecule is directly or indirectly responsible for the onset and/or the maintenance and/or the progression and/or the worsening of said disease, disorder or condition. In another particular embodiment, the target molecule is directly or indirectly responsible for specific symptoms associated with said disease, disorder or condition.

In another particular embodiment, the target molecule is directly or indirectly responsible for a lack of efficiency of therapeutic and/or prophylactic therapies against said disease, disorder or condition.

In a more particular embodiment, the target molecule is responsible for such a lack of efficiency by direct interaction with said therapeutic and/or prophylactic therapy, for example by quenching, binding, neutralizing, and/or degrading the active molecule of the therapy. In another more particular embodiment, the target molecule is responsible for such a lack of efficiency by indirect interaction with said therapeutic and/or prophylactic therapy, for example by interacting with the cells targeted by the therapy and making them non- or less responsive to the therapy.

In another particular embodiment, the target molecule is directly or indirectly responsible for specific side effects of therapeutic and/or prophylactic therapies against a disease, disorder or condition.

In a particular embodiment, the target molecule is a toxin. In particular, the target molecule may be an exotoxin or an endotoxin. Exotoxins are generated and actively secreted; endotoxins remain part of the bacteria. The response to a bacterial toxin can involve severe inflammation and can lead to sepsis.

Examples of toxins include Colibactin of E. coli, Toxin A and other enzymes (e.g., hemolysin, leukotoxin, exfoliative toxin, enterotoxin, and toxic-shock syndrome toxin-1 (TSST-1)) from Staphylococcus aureus (typically as described in Tam and Torres, Microbiol Spectr. 2019 March; 7(2)) and fragilysin (Bft) from Enterotoxigenic (ETBF) strains of Bacteroides fragilis, Botulinum neurotoxin, Tetanus toxin, Diphteria toxin, Anthrax toxin, Alpha toxin, Pertussis toxin, Shiga toxin, Heat-stable enterotoxin (E. coli ST), or any toxin described in Henkel et al., (Toxins from Bacteria in EXS. 2010; 100: 1-29).

By “colibactin” is meant herein a secondary metabolite synthetized by the clbA-S genes present in the 54-kb pathogenicity pks island, a genetic island encoding a non-ribosomal peptide synthetase-polyketide synthase (NRPS-PKS) assembly line in Enterobacteriaceae. Colibactin is typically produced as a prodrug moiety that is exported in the periplasm by the efflux pump CIbM and then hydrolyzed by the periplasmic membrane-bound ClbP protein with a peptidase activity, which releases the active colibactin.

In a particular embodiment, said method is for treating and/or preventing colorectal cancer, and said target molecule is fragilysin, in particular produced by Enterotoxigenic Bacteroides fragilis (ETBF).

In a particular embodiment, said method is for treating and/or preventing colorectal cancer, and said target molecule is colibactin, in particular produced by Enterococcus faecalis and/or E. coli.

In a particular embodiment, said target molecule is a virulence factor.

A virulence factor can be any substance produced by a pathogen that alters host-pathogen interaction by increasing the degree of damage done to the host. Virulence factors are used by pathogens in many ways, including, for example, in cell adhesion or colonization of a niche in the host, to evade the host's immune response, to facilitate entry to and egress from host cells, to obtain nutrition from the host, or to inhibit other physiological processes in the host. Virulence factors can include enzymes, endotoxins, adhesion factors, motility factors, factors involved in complement evasion, scavenging factors and factors that promote biofilm formation.

Examples of virulence factors include virulence factors encoded by the following E. coli virulence factor genes EHEC-HIyA, fimA, fimF, fimH, neuC, kpsE, sfa, foc, iroN, aer, iha, papC, papGI, papGII, papGIII, hlyC, cnf1, hra, sat, ireA, usp ompT, ibeA, malX, fyuA, irp2, traT, afaD, ipaH, eltB, estA, bfpA, eaeA, espA, aaiC, aatA, TEM, CTX, SHV, csgA, csgB, csgC, csgD, csgE, csgF, csgG, csgH, genes within T1SS, T2SS, T3SS, T4SS, T5SS, T6SS (secretion systems) and blc, virulence factors encoded by the Yersinia pestis virulence factor gene yscF, virulence factors encoded by the Francisella tularensis virulence factor gene fslA, virulence factors encoded by the Bacillus anthracis virulence factor gene pag, virulence factors encoded by the Vibrio cholera virulence factor genes ctxA, ctxB, tcpA and toxT, virulence factors encoded by the Pseudomonas aeruginosa virulence factor genes pyoverdine (e.g., sigma factor pvdS, biosynthetic genes pvdL, pvdl, pvdJ, pvdH, pvdA, pvdF, pvdQ, pvdN, pvdM, pvdO, pvdP, transporter genes pvdE, pvdR, pvdT, opmQ), siderophore pyochelin (e.g., pchD, pchC, pchB, pchA, pchE, pchF and pchG), and toxins (e.g., exoU, exoS and exoT), virulence factors encoded by the Klebsiella pneumoniae virulence factor genes fimA and cps, virulence factors encoded by the Acinetobacter baumannii virulence factor genes ptk and epsA, virulence factors encoded by the Salmonella enterica Typhi virulence factor genes MIA and ssrB, virulence factors encoded by the Fusobacterium nucleatum virulence factor genes FadA and TIGIT, and virulence factors encoded by the Bacteroides fragilis virulence factor gene bft.

In a particular embodiment, said target molecule is a molecule encoded by a Cutibacterium acnes porphyrins gene, a CAMP-factor (CAMP1, CAMP2, CAMP3, CAMP4), Hyaluronate lyase (HYL-IB/II, HYL-IA), Lipases (GehA, GehB), Haemolysins, Sialidases, Endoglycoceramidases, Endo-β-N-acetylglucosaminidase, Dermatan sulphate adhesin (DsA1, DsA2), Proline-Threonine Repeats (PTRs) or in any virulence factors included on the acne associated genomic loci 1, 2, 3(plasmid), 4 such as a tight adhesion locus (tad), Streptolysin S-associated genes (sag), nonribosomal peptide synthetases (NRPS) as described in Tomida et al. (2013) mBio 4, e00003-13).

In a particular embodiment, in particular in a method for treating and/or preventing gastric cancer, said target molecule is cytotoxin-associated antigen A (CagA) and/or vacuolating cytotoxin (VacA), preferably produced by Helicobacter pylori.

When said target molecule is a toxin or a virulence factor, as disclosed above, the molecule of interest encoded by the heterologous or engineered nucleic acid can typically be a molecule involved in the degradation, inactivation, adsorption, absorption and/or transport of said target molecule.

In a particular embodiment, said method is for treating and/or preventing hyperphenylalaninemia, and said target molecule is phenylalanine. In that embodiment, said heterologous or engineered nucleic acid typically encodes a phenylalanine ammonia lyase (PAL), a phenylalanine transporter and/or an L-amino acid deaminase (LAAD).

In another particular embodiment, said method is for treating and/or preventing disease involving propionate catabolism, and said target molecule is propionyl CoA or methylmalonyl CoA. In that embodiment, said heterologous or engineered nucleic acid typically encodes one or more propionate catabolism enzyme(s), one or more transporter(s) of propionate, and/or one or more exporter(s) of succinate.

In another particular embodiment, said method is for treating and/or preventing a disease associated with excess branched amino acid, such as MSUD, isovaleric acidemia (IV A), propionic acidemia, methylmalonic acidemia, and diabetes ketoacidosis, as well as other diseases, for example, 3-MCC Deficiency, 3-Methylglutaconyl-CoA hydratase Deficiency, HMG-CoA Lyase Deficiency, Acetyl-CoA Carboxylase Deficiency, Malonyl-CoA Decarboxylase Deficiency, short-branched chain acylCoA dehydrogenase deficiency, 2-methyl-3-hydroxybutyric acidemia, beta-ketothiolase deficiency, isobutyryl-CoA dehydrogenase deficiency, HIBCH deficiency, and 3-Hydroxyisobutyric aciduria, and said target molecule is branched amino acid such as leucine, isoleucine, or valine. In that embodiment, said heterologous or engineered nucleic acid typically encodes one or more transporter(s) of a branched chain amino acid, one or more branched chain amino acid binding protein(s), one or more branched chain amino acid catabolism enzyme(s) that are capable of converting a-ketoisocaproate, a-keto-P-methylvalerate, and/or a-ketoisovalerate to isovaleraldehyde, 2-methylbutyraldehyde, and/or isobutyraldehyde, one or more a-ketoacid decarboxylase(s), one or more branched chain amino acid catabolism enzyme(s) that are capable of converting leucine, isoleucine and/or valine to a-ketoisocaproate, a-keto-β-methylvalerate, and/or α-ketoisovalerate, one or more branched chain amino acid deamination enzymes such as a branched chain amino acid dehydrogenase, branched chain amino acid aminotransferase, and amino acid oxidase, leucine dehydrogenase, one or more branched chain amino acid catabolism enzyme(s) that are capable of converting isovaleraldehyde, isobutyraldehyde and/or 2-methylbutyraldehyde to isopentanol, isobutanol, and/or 2-methylbutanol, one or more branched chain amino acid alcohol dehydrogenases, one or more branched chain amino acid catabolism enzyme(s) that are capable of converting isovaleraldehyde, isobutyraldehyde and/or 2-methylbutyraldehyde to isovalerate, isobutyrate, and/or 2-methylbutyrate, or one or more branched chain amino acid aldehyde dehydrogenases.

In a particular embodiment, said method is for preventing and/or treating a disorder in which trimethylamine is detrimental such as a cardiovascular disease, in particular atherosclerosis, or a kidney disease, and said target molecule is trimethylamine. In that embodiment, said heterologous or engineered nucleic acid typically encodes one or more trimethylamine (TMA) catabolism enzyme(s), such as enzymes that catabolize trimethylamine (TMA) and/or trimethylamine N-oxide, and/or one or more transporter(s) of trimethylamine (TMA) and/or trimethylamine N-oxide (TMAO).

In a particular embodiment, said method is for preventing and/or treating a disease, disorder or condition associated with bile salts such as a metabolic disease (in particular diabetes or obesity) or a cardiovascular disease (in particular hypercholesterolemia), and said target molecule is bile salts. In that embodiment, said heterologous or engineered nucleic acid typically encodes a bile salt hydrolase enzyme, and/or one or more 7a-dehydroxylating enzyme(s).

In a particular embodiment, said method is for preventing and/or treating a disorder in which oxalate is detrimental, in particular hyperoxaluria, and said target molecule is oxalate. In that embodiment, said heterologous or engineered nucleic acid typically encodes one or more oxalate catabolism enzymes.

In a particular embodiment, said method is for preventing and/or treating a disease, disorder or condition caused by a toxic molecule, metabolite or other deleterious molecule, such as a chemotherapy-induced diarrhea or gastrointestinal toxicity, a NSAID-induced diarrhea or gastrointestinal toxicity, or heavy metal poisoning, and said target molecule is a chemotherapeutic drug (in particular selected from irinotecan; methotrexate; an antimetabolite such as gemcitabine and cytosine arabinoside; a fluoropyrimidine such as fluorouracil, capecitabine, and tegafur/uracil; a multitargeted folinic acid antagonist such as pemetrexed, raltitrexed, and gemcitabine; a plant alkaloid; a vinca alkaloid such as vincristine and vinorelbine; a epipodophyllotoxin such as etoposide; a taxane such as paclitaxel and docetaxel; a topoisomerase I inhibitor; a cytotoxic antibiotic; an anthracycline such as doxorubicin, daunorubicin, idarubicin, aclarubicin, and daunomycin; an alkylating agent such as cyclophosphamide; a platinum such as cisplatin, carboplatin, oxaliplatin and nedaplatin; an antibody such as ipilumumab; an antibody against VEGF such as bevacizumab; a tyrosine-kinase inhibitor; an EGFR inhibitor such as lapatinib and cetuximab; and a metabolite or byproduct thereof), a NSAID (in particular selected from naproxen, indomethacin, ketoprofen, piroxicam, ibuprofen, diclofenac, a COX-2 inhibitor, and a metabolite or byproduct thereof), a heavy metal (in particular elected from aluminum, antimony, arsenic, barium, bismuth, cadmium, chromium, cobalt, copper, gold, iron, lead, lithium, manganese, mercury, nickel, phosphorous, platinum, selenium, silver, thallium, tin, and zinc), or a metabolite or byproduct thereof. In that embodiment, said heterologous or engineered nucleic acid typically encodes a molecule that is capable of detoxifying said deleterious molecule.

In a particular embodiment, the target molecule is a bacterial enzyme.

In a more particular embodiment, the target molecule is a bacterial enzyme targeting a drug administered to the subject to treat and/or prevent a disease, disorder or condition. In the context of the invention, a “bacterial enzyme targeting a drug” encompasses both an enzyme leading to the elimination, deactivation or reactivation of the drug and an enzyme involved in the more general evolution of the drug activity in the subject once administered to said subject.

Said given drug may be selected from the group consisting of Nicardipine.HCl, Risperidone, Tolcapone, Azathioprine, Entacapone, Exemestane, Nimodipine, Capsaicin, Dexamethasone, Ethacrynic Acid, Rifampin (Rifampicin), Sulindac, Vorinostat, Dolasetron, Mycophenolate Mofetil, Zidovudine (3′-Azido-3′-Deoxythymidine), Allopurinol, Betamethasone, Bisacodyl, Estradiol, Famciclovir, Flutamide, Hydrocortisone, Hydrocortisone Acetate, Methylprednisolone, Metronidazole, Nabumetone, Pantoprazole, Prednisolone, Progesterone, Prednisone, Spironolactone, Sulfasalazine, Tinidazole, Fluoxetine.HCl, Misoprostol, Megestrol Acetate, Capecitabine, Chenodiol (Chenodeoxycholic Acid), Clofazimine, Clonazepam, Cortisone Acetate, Dantrolene-Na, Duloxetine, Fludrocortisone Acetate, Iloperidone, Lorazepam, Nilutamide, Nitisinone, Nitrofurantoin, Oxazepam, Paliperidone, Prasugrel, Probenecid, Rifabutin, Sulfamethoxazole, Ursodiol, Omeprazole, Tenatoprazole, Artemisinin, Danazol, Olmesartan medoxomil, Phenazopyridine, Nitrendipine, Racecadrotil, Fenofibrate, Fluphenazine, Telmisartan, Benzbromarone, Oxethazaine, Mefloquine, Quinacrine, Pimozide, Loxapine succinate, Cyclobenzaprine, Ethopropazine, Promethazine, Clemizole and Pyrimethamine.

Said given drug may further be selected from the group consisting of abacavir sulfate, acebutolol, acecainide, alfuzosin, almotriptan, alprenolol, amantadine, aminoglutethimide, amisulpride, anagrelide, anastrozole, antazoline phosphate, apomorphine, artemisinin, atenolol, atorvastatin calcium, azatadine maleate, bambuterol, Benazepril, benzbromarone, benzthiazide, betamethasone acetate, betamethasone valerate, betaxalol, bezafibrate, bicalutamide, biperiden, bisacodyl, bisoprolol fumarate, bromocriptine mesylate, budesonide, bupropion, buramate, buspirone, camylofine dihydrochloride, capecitabine, carbetapentane citrate, carbinoxamine maleate, carisoprodol, Carvedilol, celecoxib, cetirizine, chlormezanone, cimetidine, citalopram hydrobromide, clemastine fumarate, clemizole, clenbuterol, clidinium bromide, clonidine, clopidogrel sulfate, Clozapine, colchicine, cyclobenzaprine, cyclophosphamide, cyproterone acetate, dabigatran etexilate mesylate, danazol, darifenacin hydrobromide, dasatinib, deflazacort, desvenlafaxine succinate, dexamethasone, dextromethorphan hydrobromide, diacetamate, Dicyclomine, diflorasone diacetate, digitoxin, digoxin, diltiazem, diperodon, diphenylpyraline, Dipyridamole, disopyramide phosphate, domperidone, doxazosin mesylate, doxepin, doxylamine succinate, drospirenone, duloxetine, eletriptan hydrobromide, enalapril maleate, Entacapone, ergonovine maleate, ergotamine tartrate, eszopiclone, ethopropazine, ethoxzolamide, ethynodiol diacetate, etodolac, ezetimibe, famciclovir, famprofazone, febuxostat, fenofibrate, fenspiride, fexofenadine, finasteride, fluconazole, fluoxetine, fluphenazine, fluvoxamine maleate, galantamine, gliclazide, glipizide, griseofulvin, guanfacine, haloperidol, hyoscyamine, idebenone, imatinib, indapamide, indomethacin, Irbesartan, irsogladine maleate, isradipine, itraconazole, ketorolac tromethamine, ketotifen fumarate, labetalol, lamotrigine, letrozole, levamisole, levonorgestrel, linagliptin, lofexidine, Loperamide, losartan, lovastatin, loxapine succinate, mebendazole, mebhydrolin naphthalenesulfonate, mefloquine, megestrol acetate, melphalan, memantine, metaxalone, Methocarbamol, methoxsalen, methsuximide, methylphenidate, methysergide maleate, meticrane, metitepine maleate, metoclopramide, metolazone, metoprolol tartrate, mevastatin, mianserin, mifepristone, milnacipran, mycophenolate mofetil, nadolol, nafronyl oxalate, naftopidil, naloxone, naproxen(+), nateglinide, nefazodone, nefopam, neostigmine bromide, nevirapine, nicergoline, nitrendipine, nizatidine, norethindrone acetate, Norgestimate, noscapine, olanzapine, olmesartan medoxomil, omeprazole, orphenadrine citrate, oxaprozin, oxcarbazepine, oxethazaine, oxybutynin chloride, Paclitaxel, paliperidone, pantoprazole, papaverine, paroxetine, penbutolol sulfate, pentoxifylline, pergolide mesylate, pericyazine, perindopril erbumine, phenacetin, Phenazopyridine, phenytoin sodium, pidotimod, pimozide, pitavastatin calcium, Pranoprofen, prazosin, prednisone, pridinol methanesulfonate, primaquine phosphate, Procarbazine, promethazine, pyrimethamine, quetiapine, quinacrine, quinapril, quinine sulfate, racecadotril, ramelteon, ramipril, ranitidine, ranolazine, rebamipide, repaglinide, Reserpine, riluzole, rimantadine, risperidone, ritonavir, rivastigmine tartrate, rizatriptan benzoate, ropinirole, rosiglitazone maleate, rosuvastatin calcium, roxatidine acetate, sertraline, sildenafil citrate, solifenacin succinate, sotalol, spiperone, sulfasalazine, sulfinpyrazone, sulindac, sulpiride, sumatriptan succinate, tacrine, tacrolimus, tadalafil, tamsulosin hydrchloride, tegaserod maleate, telmisartan, tenatoprazole, tenoxicam, terazosin, terbinafine, thiabendazole, thiothixene, tiapride, timolol maleate, tinidazole, Tolazamide, topotecan, trandolapril, tranilast, trazodone, trihexyphenidyl, trimebutine maleate, trimetazidine dihydrochloride, trimethobenzamide, trimipramine maleate, Triprolidine, tropisetron, trospium chloride, valsartan, venlafaxine, verapamil, vilazodone, Vinpocetine, voriconazole, warfarin, zaleplon, zidovudine [azt], ziprasidone mesylate and zolpidem.

In a particular embodiment, said target molecule is selected from the bacterial enzymes having oxidation, deamination, isomerization, esterification, condensation, reduction, hydrolysis and/or rearrangement activities. In a particular embodiment, said target molecule is selected from p-glucuronidases, nitroreductases and sulfoxide reductases.

In a particular embodiment, when said given drug is dantrolene, clonazepam, and/or nicardipine, said target molecule is an enzyme having nitro-reduction activity.

In an alternative embodiment, when said given drug is risperidone, said target molecule is an enzyme having hydrolysis activity, in particular an enzyme hydrolysing the isoxazole moiety of risperidone.

In an alternative embodiment, when said given drug is sulfasalazine, said target molecule is an enzyme having azoreduction activity.

In an alternative embodiment, when said given drug is digoxin, said target molecule is cytochrome glycoside reductase.

In an alternative embodiment, when said given drug is levodopa (L-DOPA), in particular in a subject suffering from Parkinson's disease, said target molecule is tyrosine decarboxylase and/or dopamine dehydrolase.

In another embodiment, when said given drug is levodopa (L-DOPA), in particular in a subject suffering from Parkinson's disease, said target molecule is DHPAA synthase.

In an alternative embodiment, when said given drug is gemcitabine, said molecule is a cytidine deaminase.

In an alternative embodiment, when said given drug is prontosil, said target molecule is an enzyme converting prontosil into p-aminobenzenesulfonamide by azo-reduction.

In an alternative embodiment, when said given drug is selected from sulfasalazine, ipsalazide and balsalazide, said target molecule is an enzyme converting said drug into 5-aminosalicylic acid.

In an alternative embodiment, when said given drug is a non-steroidal anti-inflammatory drug, said target molecule is a p-glucuronidase.

In a particular embodiment, in particular in a method for treating and/or preventing type 2 diabetes, said target molecule is a bacterial enzyme involved in amino acid metabolism.

In a particular embodiment, said target molecule is a bacterial adhesin.

In a more particular embodiment, said target molecule is a bacterial adhesin involved in the adsorption of a given drug, administered to the subject to treat and/or prevent a disease, disorder or condition, to a bacterial strain.

In a particular embodiment, when said given drug is L-DOPA, typically in a subject suffering from Parkinson's disease, said target molecule is a molecule involved in the adsorption of L-DOPA by Helicobacter pylori, typically a bacterial adhesin from H. pylori involved in said adsorption.

In a particular embodiment, said target molecule is a bacterial molecule competing with a given drug for a receptor of the subject's cells.

In a particular embodiment, when said given drug is acetaminophen, said target molecule is p-cresol produced by C. difficile or an enzyme involved in the production and/or secretion of p-cresol by C. difficile.

The present invention also concerns a method for preventing and/or treating, in a subject, a disease, disorder or condition, a therapy of which comprises a target molecule, said method comprising administering to the subject a therapeutically efficient amount of (a) an engineered bacterial strain, as defined above and (b) a milk oligosaccharide,
wherein said engineered bacterial strain comprises:
   (i) an heterologous or engineered nucleic acid involved in the expression of a molecule of interest, wherein the expression of said molecule of interest directly or indirectly increases the level of the target molecule in said subject, and
   (ii) an autologous gene or gene set involved in the import and/or metabolism of a milk oligosaccharide,
whereby the disease, disorder or condition, a therapy of which comprises the target molecule, is prevented or treated.

The present invention also concerns a composition or combination comprising an engineered bacterial strain and milk oligosaccharide for use in a method for preventing and/or treating, in a subject, a disease, disorder or condition, a therapy of which comprises a target molecule, wherein said engineered bacterial strain comprises:
(i) an heterologous or engineered nucleic acid involved in the expression of a molecule of interest, wherein the expression of said molecule of interest directly or indirectly increases the level of the target molecule in said subject, and
(ii) an autologous gene or gene set involved in the import and/or metabolism of a milk oligosaccharide.

The present invention also concerns the use of an engineered bacterial strain and a milk oligosaccharide for the manufacture of a medicament or a pharmaceutical combination intended for the prevention and/or the treatment, in a subject, of a disease, disorder or condition, a therapy of which comprises a target molecule, wherein said engineered bacterial strain comprises:
(i) an heterologous or engineered nucleic acid involved in the expression of a molecule of interest, wherein the expression of said molecule of interest directly or indirectly increases the level of the target molecule in a microbiome of said subject, and
(ii) an autologous gene or gene set involved in the import and/or metabolism of a milk oligosaccharide.

In a particular embodiment, said method is for preventing and/or treating a disease, disorder or condition that benefit from reduced gut inflammation and/or tighten gut mucosal barrier, such as an autoimmune disorder (in particular selected from the group consisting of type 1 diabetes, asthma, multiple sclerosis, lupus, rheumatoid arthritis, ulcerative colitis, juvenile arthritis, psoriasis, psoriatic arthritis, Crohn's disease, celiac disease, and ankylosing spondylitis) or a disease or condition associated with gut inflammation and/or compromised gut barrier function (in particular selected from an inflammatory bowel disease and a diarrheal disease). In that embodiment, said heterologous or engineered nucleic acid typically encodes a non-native, anti-inflammatory molecule, a non-native gut barrier function enhancer molecule and/or a biosynthetic pathway, wherein the final product of the biosynthetic pathway is an anti-inflammatory molecule and/or a gut barrier function enhancer molecule.

In a particular embodiment, said method is for preventing and/or treating a disease, disorder or condition selected from the group consisting of Nonalcoholic Steatohepatitis (NASH), Nonalcoholic fatty liver disease (NAFLD), type 1 diabetes, type 2 diabetes, metabolic syndrome, Bardet-Biedel syndrome, Prader-Willi syndrome, tuberous sclerosis, Albright hereditary osteodystrophy, brain-derived neurotrophic factor (BDNF) deficiency; Single-minded 1 (SIM1) deficiency, leptin deficiency, leptin receptor deficiency, pro-opiomelanocortin (POMC) defects, proprotein convertase subtilisin/kexin type 1 (PCSK1) deficiency, Src homology 2B 1 (SH2B 1) deficiency, pro-hormone convertase 1/3 deficiency, melanocortin-4-receptor (MC4R) deficiency, Wilms tumor, aniridia, genitourinary anomalies, mental retardation (WAGR) syndrome, pseudohypoparathyroidism type 1A, Fragile X syndrome, Borjeson-Forsmann-Lehmann syndrome, Alstrom syndrome, Cohen syndrome and ulnar-mammary syndrome, and said target molecule is a gut barrier enhancer molecule or a satiety effector molecule. In that embodiment, said heterologous or engineered nucleic acid is typically a gene or gene cassette for producing a gut barrier enhancer molecule, a gene or gene cassette for producing a satiety effector molecule, or encodes one or more enzymes present in a biosynthetic pathway for producing a short chain fatty acid, e.g., butyrate, propionate, and/or acetate.

In another particular embodiment, said method is for treating and/or preventing a metabolic disease, such as obesity or type 2 diabetes, and said target molecule is a metabolic or satiety effector molecule, preferably selected from the group consisting of n-acyl-phosphatidylethanolamine (NAPE), a n-acyl-ethanolamines (NAE), a ghrelin receptor antagonist, peptide YY3-36, a cholecystokinin (CCK), CCK58, CCK33, CCK22, CCK8, a bombesin, gastrin releasing peptide (GRP), neuromedin B (P), glucagon, GLP-1, GLP-2, apolipoprotein A-IV, amylin, somatostatin, enterostatin, oxyntomodulin, pancreatic peptide, a short-chain fatty acid, butyrate, propionate, acetate, a serotonin receptor agonist, nicotinamide adenine dinucleotide (NAD), nicotinamide mononucleotide (NMN), nucleotide riboside (NR), nicotinamide, and nicotinic acid (NA), and/or tryptophan metabolite. In that embodiment, said heterologous or engineered nucleic acid typically encodes a tryptophan transporter, an enzyme metabolizing tryptophan, an enzyme for producing a tryptophan metabolite, an enzyme for producing kynurenine, an enzyme for producing kynurenic acid, an enzyme for producing an indole, an enzyme for producing indole-3-acetic acid, or any of the above target molecules.

In another particular embodiment, said method is for treating and/or preventing a cancer, typically selected from adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, largyngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer, lymphoma (e.g., AIDS-related lymphoma, Burkitt lymphoma, cutaneous T cell lymphoma, Hogkin lymphoma, Non-Hogkin lymphoma, primary central nervous system lymphoma), malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenström macroglobulinemia, and Wilms tumor; and said target molecule is an anti-cancer molecule. In that embodiment, said heterologous or engineered nucleic acid is typically a gene sequence(s) or gene set for producing one or more anti-cancer molecule(s) such as one or more immune checkpoint inhibitor(s) (in particular selected from a CTLA-4 inhibitor, a PD-1 inhibitor, and a PD-L1 inhibitor), an immune checkpoint inhibitor of TIGIT, VISTA, LAG-3, TIM1, CEACAM1, LAIR-1, HVEM, BTLA, CD160, CD200, CD200R, GITR, orA2aR, IL-15, IL-12, IL-2, GM-CSF, IL-21, an agonist ligand for OX40, an agonist ligand for ICOS, kynureninase, arginine, a cytotoxin or a lytic peptide.

In a particular embodiment, the method of the invention is for improving menopause symptoms. In such embodiment, said engineered bacterial strain preferably comprises at least one heterologous nucleic acid encoding a daidzein-metabolizing enzyme, more particularly at least one heterologous nucleic acid encoding a daidzein-to-dihydrodaidzein (DHD)-converting enzyme and/or at least one heterologous nucleic acid encoding a DHD-to-equol-converting enzyme. In a more particular embodiment, said engineered bacterial strain comprises at least one heterologous nucleic acid selected from the group consisting of the orf-1 and orf-2 genes from Slackia sp. strain NATTS (as disclosed in Tsuji et al. (2012) Appl Environ Microbiol. 78(4): 1228-1236), and/or at least one heterologous nucleic acid which is the orf-3 gene from Slackia sp. strain NATTS (as disclosed in Tsuji et al. (2012) Appl Environ Microbiol. 78(4): 1228-1236).

In another particular embodiment, the method of the invention is for improving gluten intolerance.

In another particular embodiment, the method of the invention is for improving lactose intolerance.

In another particular embodiment, the method of the invention is for decreasing microbiome-induced irinotecan toxicity.

In another particular embodiment, the method of the invention is for treating and/or preventing phenylketonuria (PKU).

In another particular embodiment, the method of the invention is for treating and/or preventing enteric hyperoxaluria.

In the context of the invention, examples of diseases, disorders or conditions to be treated or prevented by the method of the invention include a neurodegenerative disease or condition; a brain disease or condition; a CNS disease or condition; memory loss or impairment; a heart or cardiovascular disease or condition, such as heart attack, stroke or atrial fibrillation; a liver disease or condition; a kidney disease or condition, such as chronic kidney disease (CKD); a pancreas disease or condition; a lung disease or condition, such as cystic fibrosis or COPD; a gastrointestinal disease or condition; a throat or oral cavity disease or condition; an ocular disease or condition; a genital disease or condition, such as a vaginal, labial, penile or scrotal disease or condition; a sexually-transmissible disease or condition, such as gonorrhea, HIV infection, syphilis or chlamydia infection; an ear disease or condition; a skin disease or condition; a heart disease or condition; a nasal disease or condition; a haematological disease or condition, such as anaemia, in particular anaemia of chronic disease or cancer; a viral infection; a pathogenic bacterial infection; a cancer; an autoimmune disease or condition, such as SLE; an inflammatory disease or condition, such as rheumatoid arthritis, psoriasis, eczema, asthma, ulcerative colitis, colitis, Crohn's disease or IBD; autism; ADHD; bipolar disorder; ALS (Amyotrophic Lateral Sclerosis); osteoarthritis; a congenital or development defect or condition; miscarriage; a blood clotting condition; bronchitis; dry or wet AMD; neovascularisation (for example of a tumour or in the eye); common cold; epilepsy; fibrosis, such as, liver or lung fibrosis; a fungal disease or condition, such as thrush; a metabolic disease or condition, such as obesity, anorexia, diabetes, Type I or Type II diabetes; ulcer(s), such as gastric ulceration or skin ulceration; dry skin; Sjogren's syndrome; cytokine storm; deafness, hearing loss or impairment; slow or fast metabolism (ie, slower or faster than average for the weight, sex and age of the subject); conception disorder, such as infertility or low fertility; jaundice; skin rash; Kawasaki disease; Lyme disease; an allergy, such as a nut, grass, pollen, dust mite, cat or dog fur or dander allergy; malaria, typhoid fever, tuberculosis or cholera; depression; mental retardation; microcephaly; malnutrition; conjunctivitis; pneumonia; pulmonary embolism; pulmonary hypertension; a bone disorder; sepsis or septic shock; sinusitus; stress (such as occupational stress); thalassaemia, anaemia, von Willebrand Disease, or haemophilia; Shingles or cold sore; menstruation; and low sperm count.

In an example, the neurodegenerative or CNS disease, disorder or condition is selected from the group consisting of Alzheimer disease, geriopsychosis, Down syndrome, Parkinson's disease, Creutzfeldt-Jakob disease, diabetic neuropathy, Parkinson syndrome, Huntington's disease, Machado-Joseph disease, amyotrophic lateral sclerosis and diabetic neuropathy.

Autoimmune diseases that may be treated or prevented include Acute Disseminated Encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, Lambert-Eaton syndrome, leukocytoclastic vasculitis, lichen planus, lichen sclerosus, ligneous conjunctivitis, linear IgA disease (LAD), lupus (SLE), Lyme disease, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonage-Turner syndrome, Pars planitis (peripheral uveitis), pemphigus, peripheral neuropathy, perivenous encephalomyelitis, pernicious anemia, POEMS syndrome, polyarteritis nodosa, Tolosa-Hunt syndrome, transverse myelitis, Type 1 diabetes, ulcerative colitis, undifferentiated connective tissue disease (UCTD), uveitis, vasculitis, vesiculobullous dermatosis, vitiligo, and Wegener's granulomatosis (now termed Granulomatosis with Polyangiitis (GPA).

Inflammatory diseases that may be treated or prevented include Alzheimer disease, ankylosing spondylitis, arthritis (osteoarthritis, rheumatoid arthritis (RA), psoriatic arthritis), asthma, atherosclerosis, Crohn's disease, colitis, dermatitis, diverticulitis, fibromyalgia, hepatitis, irritable bowel syndrome (IBS), systemic lupus erythematosus (SLE), nephritis, Parkinson's disease, and ulcerative colitis.

In the context of the invention, the term “treating” or “treatment” means reversing, alleviating, or inhibiting the progress of the disease, disorder or condition to which such term applies, or one or more symptoms of such disease, disorder or condition.

In the context of the invention, the term “prevention” refers to any indicia of success in protecting a subject or patient (e.g. a subject or patient at risk of developing a disease or condition) from developing, contracting, or having a disease, disorder or condition, including preventing one or more symptoms of a disease, disorder or condition or diminishing the occurrence, severity, or duration of any symptoms of a disease, disorder or condition following administration of the engineered bacterial strain as described herein.

By a “therapeutically effective amount” of an engineered bacterial strain of the invention and milk oligosaccharide is meant a sufficient amount of the engineered bacterial strain and the milk oligosaccharide to treat a specific disease, disorder or condition, to contribute to the treatment of a specific disease, disorder or condition, or to avoid side effects of a treatment of a specific disease, disorder or condition, at a reasonable benefit/risk ratio applicable to any medical treatment. It will be understood, however, that the total daily usage of the engineered bacterial strain of the present invention and milk oligosaccharide will be decided by the attending physician within the scope of sound medical judgment. The specific therapeutically effective dose level for any particular subject will depend upon a variety of factors including the disease, disorder or condition being treating and the severity of the disease, disorder or condition, activity of the specific engineered bacterial strain and milk oligosaccharide employed, the specific combinations employed, the age, body weight, general health, sex and diet of the subject, the time of administration, route of administration and rate of excretion of the specific engineered bacterial strains and milk oligosaccharide employed, the duration of the treatment, drugs used in combination or coincidental with the specific engineered bacterial strains and milk oligosaccharide employed, and like factors well known in the medical arts.

The present invention also concerns a method for the cosmetic caring of a subject presenting an unesthetic manifestation due to a target molecule, said method comprising
administering to the subject a cosmetically efficient amount of an engineered bacterial strain, comprising:
   (i) an heterologous or engineered nucleic acid involved in the expression of a molecule of interest wherein the expression of the molecule of interest directly or indirectly decreases the level of or modifies the target molecule in said subject, and
   (ii) an autologous gene or gene set involved in the import and/or metabolism of a milk oligosaccharide, as defined above, and
further administering to said subject said milk oligosaccharide.

The present invention also concerns a method for the cosmetic caring of a subject presenting an unesthetic manifestation which can be treated by a target molecule, said method comprising:
administering to the subject a cosmetically efficient amount of an engineered bacterial strain, comprising
   (i) an heterologous or engineered nucleic acid involved in the expression of a molecule of interest wherein the expression of the molecule of interest directly or indirectly increases the amount of the target molecule in said subject, and
   (ii) an autologous gene or gene set involved in the import and/or metabolism of a milk oligosaccharide, as defined above, and
further administering to said subject said milk oligosaccharide.

By “unesthetic manifestation” is meant herein a non-pathological manifestation on a subject, in particular on the skin of a subject, of the effect of a particular molecule on the subject. Examples of unesthetic manifestation include redness, feeling of heat or warmth, tension, tingling, stinging, tightness, pigment spots, burning sensation, itching sensation, tautness, visible squama, thickening of the skin, wrinkles, sagging skin, localized resistant fat, and/or cellulite appearance.

In both cosmetic methods, the engineered bacterial strain becomes present in the microbiome of said subject at a colonization level enabling an overall production of the molecule of interest in an amount efficient for modulating the level of or modifying the target molecule at a rate leading to a cosmetic effect on said subject.

The methods of the invention of modulating the level of or modifying a target molecule can also be applied to the environment.

For example, the method of the invention can be applied to soil or water to eliminate a toxin or environmental contamination, such as in an industrial chemical spill or waste product. The method of the invention can also be applied to waste water or industrial waste or byproduct to decontaminate or detoxify the waste. In yet other embodiments, the method of the invention can be applied to industrial or environmental material such as but not limited to agricultural or food production waste to produce or improve the production of a metabolic product.

### Product and Composition

The present invention also concerns an engineered bacterial strain, as defined above, comprising an heterologous or engineered nucleic acid involved in the expression of the molecule of interest, as defined above, and an autologous gene or gene set involved in the import and/or metabolism of a milk oligosaccharide, as defined above.

In particular embodiments, the engineered bacterial strain of the invention is in a pharmaceutical, veterinary or cosmetic composition.

The pharmaceutical or veterinary composition according to the invention may further comprise a pharmaceutically acceptable excipient.

By “pharmaceutically acceptable excipient” is meant herein a non-pharmaceutically active additive used in the manufacture of a pharmaceutical composition, which allows the pharmaceutically active ingredient to be manufactured into a pharmaceutical formulation or a galenic formulation providing the necessary bioavailability of the medicament to the patient upon the administration of the pharmaceutical composition. The excipient is preferably compatible with the other ingredients of the composition and produces no adverse effect, allergic reaction or other undesirable reaction when it is administered to a human or an animal.

The cosmetic composition according to the invention may further comprise a cosmetically acceptable excipient.

By “cosmetically acceptable excipient” is meant herein a substance that is non-toxic, biologically tolerable, and otherwise biologically suitable for administration to a subject, such as an inert substance, added to a cosmetic composition or otherwise used as a vehicle, carrier, or diluents to facilitate administration of a cosmetically active ingredient and that is compatible therewith.

A solid pharmaceutically or cosmetically acceptable vehicle or excipient may include one or more substances which may also act as flavoring agents, lubricants, solubilizers, suspending agents, dyes, fillers, glidants, compression aids, inert binders, sweeteners, preservatives, dyes, coatings, or tablet-disintegrating agents. Suitable solid vehicles include, for example calcium phosphate, magnesium stearate, talc, sugars, lactose, dextrin, starch, gelatin, cellulose, polyvinylpyrrolidone, low melting waxes and ion exchange resins.

The pharmaceutical, veterinary or cosmetic composition may be prepared as a sterile solid composition that may be suspended at the time of administration using sterile water, saline, or other appropriate sterile injectable medium. The pharmaceutical, veterinary or cosmetic compositions of the invention may be administered orally in the form of a sterile solution or suspension containing other solutes or suspending agents (for example, enough saline or glucose to make the solution isotonic), bile salts, acacia, gelatin, sorbitan monooleate, polysorbate 80 (oleate esters of sorbitol and its anhydrides copolymerized with ethylene oxide) and the like. The engineered bacterial strain according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for enteral administration include sterile solutions, emulsions, and suspensions.

The engineered bacterial strain according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical or cosmetic additives such as solubilizers, emulsifiers, buffers, preservatives, sweeteners, flavoring agents, suspending agents, thickening agents, colors, viscosity regulators, stabilizers or osmo-regulators. Suitable examples of liquid vehicles for oral and enteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for enteral administration. The liquid vehicle for pressurized compositions can be a halogenated hydrocarbon or other pharmaceutically acceptable propellant.

In some embodiments, the invention encompasses pharmaceutical, veterinary or cosmetic composition formulated for delayed or gradual enteric release. In preferred embodiments, formulations or pharmaceutical preparations of the invention are formulated for delivery of the engineered bacterial strain into the distal small bowel and/or the colon. The formulation can allow the engineered bacterial strain to pass through stomach acid and pancreatic enzymes and bile, and reach undamaged to be viable in the distal small bowel and colon.

In some embodiments, the pharmaceutical, veterinary or cosmetic composition is micro-encapsulated, formed into tablets and/or placed into capsules, preferably enteric-coated capsules.

In some embodiments, the pharmaceutical, veterinary or cosmetic compositions are formulated for delayed or gradual enteric release, using cellulose acetate (CA) and polyethylene glycol (PEG). In some embodiments, the pharmaceutical, veterinary or cosmetic compositions are formulated for delayed or gradual enteric release using a hydroxypropylmethylcellulose (HPMC), a microcrystalline cellulose (MCC) and magnesium stearate. In some embodiments, the pharmaceutical, veterinary or cosmetic compositions are formulated for delayed or gradual enteric release using e.g., a poly(meth)acrylate, e.g. a methacrylic acid copolymer B, a methyl methacrylate and/or a methacrylic acid ester, or a polyvinylpyrrolidone (PVP).

In some embodiments, the pharmaceutical, veterinary or cosmetic compositions are formulated for delayed or gradual enteric release using a release-retarding matrix material such as: an acrylic polymer, a cellulose, a wax, a fatty acid, shellac, zein, hydrogenated vegetable oil, hydrogenated castor oil, polyvinylpyrrolidone, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a co-polymer of lactic and glycolic acid, carboxymethyl starch, potassium methacrylate/divinylbenzene copolymer, crosslinked polyvinylpyrrolidone, polyvinylalcohols, polyvinylalcohol copolymers, polyethylene glycols, non-crosslinked polyvinylpyrrolidone, polyvinyl acetates, polyvinylacetate copolymers or any combination thereof.

In some embodiments, the pharmaceutical, veterinary or cosmetic compositions are formulated for delayed or gradual enteric release as described in U.S. Pat. App. Pub. 20110218216, which describes an extended release pharmaceutical composition for oral administration, and uses a hydrophilic polymer, a hydrophobic material and a hydrophobic polymer or a mixture thereof, with a microenvironment pH modifier. The hydrophobic polymer can be ethylcellulose, cellulose acetate, cellulose propionate, cellulose butyrate, methacrylic acid-acrylic acid copolymers or a mixture thereof. The hydrophilic polymer can be polyvinylpyrrolidone, hydroxypropylcellulose, methylcellulose, hydroxypropylmethyl cellulose, polyethylene oxide, acrylic acid copolymers or a mixture thereof. The hydrophobic material can be a hydrogenated vegetable oil, hydrogenated castor oil, carnauba wax, candelilla wax, beeswax, paraffin wax, stearic acid, glyceryl behenate, cetyl alcohol, cetostearyl alcohol or and a mixture thereof. The microenvironment pH modifier can be an inorganic acid, an amino acid, an organic acid or a mixture thereof. Alternatively, the microenvironment pH modifier can be lauric acid, myristic acid, acetic acid, benzoic acid, palmitic acid, stearic acid, oxalic acid, malonic acid, succinic acid, adipic acid, sebacic acid, fumaric acid, maleic acid; glycolic acid, lactic acid, malic acid, tartaric acid, citric acid, sodium dihydrogen citrate, gluconic acid, a salicylic acid, tosylic acid, mesylic acid or malic acid or a mixture thereof.

In some embodiments, the pharmaceutical, veterinary or cosmetic compositions are a powder that can be included into a tablet or a suppository. In alternative embodiments, a formulation or pharmaceutical preparation of the invention can be a “powder for reconstitution” as a liquid to be drunk or otherwise administered.

In some embodiments, the pharmaceutical, veterinary or cosmetic compositions can be administered in a cream, gel, lotion, liquid, feed, or aerosol spray. Engineered bacterial strains may be immobilized onto appropriately sized polymeric beads so that the coated beads may be added to aerosols, creams, gels or liquids. The size of the polymeric beads may be from about 0.1 μm to 500 μm, for example 50 μm to 100 μm. The coated polymeric beads may be incorporated into animal feed, including pelleted feed and feed in any other format, incorporated into any other edible device used to present phage to the animals, added to water offered to animals in a bowl, presented to animals through water feeding systems. In some embodiments, the compositions are used for treatment of surface wounds and other surface infections using creams, gels, aerosol sprays and the like.

In some embodiments, the pharmaceutical, veterinary or cosmetic compositions can be administered by inhalation, in the form of a suppository or pessary, topically (e.g., in the form of a lotion, solution, cream, ointment or dusting powder), epi- or transdermally (e.g., by use of a skin patch), orally (e.g., as a tablet, which may contain excipients such as starch or lactose), as a capsule, ovule, elixirs, solutions, or suspensions (each optionally containing flavoring, coloring agents and/or excipients), or they can be injected parenterally (e.g., intravenously, intramuscularly or subcutaneously). For parenteral administration, the compositions may be used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

In some embodiments, the pharmaceutical, veterinary or cosmetic compositions can also be dermally or transdermally administered. For topical application to the skin, the pharmaceutical, veterinary or cosmetic composition can be combined with one or a combination of carriers, which can include but are not limited to, an aqueous liquid, an alcohol base liquid, a water soluble gel, a lotion, an ointment, a nonaqueous liquid base, a mineral oil base, a blend of mineral oil and petrolatum, lanolin, liposomes, proteins carriers such as serum albumin or gelatin, powdered cellulose carmel, and combinations thereof. A topical mode of delivery may include a smear, a spray, a bandage, a time-release patch, a liquid-absorbed wipe, and combinations thereof. The pharmaceutical, veterinary or cosmetic composition can be applied to a patch, wipe, bandage, etc., either directly or in a carrier(s). The patches, wipes, bandages, etc., may be damp or dry, wherein the engineered bacterial strain is in a lyophilized form on the patch. The carriers of topical compositions may comprise semi-solid and gel-like vehicles that include a polymer thickener, water, preservatives, active surfactants, or emulsifiers, antioxidants, sun screens, and a solvent or mixed solvent system. U.S. Pat. No. 5,863,560 discloses a number of different carrier combinations that can aid in the exposure of skin to a medicament, and its contents are incorporated herein.

For intranasal or administration by inhalation, the pharmaceutical, veterinary or cosmetic composition is conveniently delivered in the form of a dry powder inhaler or an aerosol spray presentation from a pressurized container, pump, spray, or nebuliser with the use of a suitable propellant, e.g., dichlorodifluoromethane, trichlorofluoromethane, dichlorotetrafluoroethane, a hydrofluoroalkane such as 1,1,1,2-tetrafluoroethane or 1,1,1,2,3,3,3-heptafluoropropane, carbon dioxide, or other suitable gas. In the case of a pressurized aerosol, the dosage unit may be determined by providing a valve to deliver a metered amount. The pressurized container, pump, spray, or nebuliser may contain a solution or suspension of the active compound, e.g., using a mixture of ethanol and the propellant as the solvent, which may additionally contain a lubricant, e.g., sorbitan trioleate. Capsules and cartridges (made, for example, from gelatin) for use in an inhaler or insufflator may be formulated to contain a powder mix of the engineered bacterial strain of the invention and a suitable powder base such as lactose or starch.

For administration in the form of a suppository or pessary, the pharmaceutical, veterinary or cosmetic composition can be applied topically in the form of a gel, hydrogel, lotion, solution, cream, ointment, or dusting powder. Compositions of the invention may also be administered by the ocular route. For ophthalmic use, the compositions of the invention can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

Dosages and desired drug concentrations of the pharmaceutical and veterinary composition compositions of the present invention may vary depending on the particular use. The determination of the appropriate dosage or route of administration is within the skill of an ordinary physician. Animal experiments can provide reliable guidance for the determination of effective doses in human therapy.

For transdermal administration, the pharmaceutical, veterinary or cosmetic composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compounds can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

### Subject, Regimen and Administration

The subject according to the invention is an animal, preferably a mammal, even more preferably a human. However, the term “subject” can also refer to non-human animals, in particular mammals such as dogs, cats, horses, cows, pigs, sheep, donkeys, rabbits, ferrets, gerbils, hamsters, chinchillas, rats, mice, guinea pigs and non-human primates, among others, or non-mammals such as poultry, that are in need of treatment.

The human subject according to the invention is preferably an adult at any age.

In a preferred embodiment, the subject has been diagnosed with, or is at risk of developing a disease, disorder or condition due to or associated with the target molecule or a disease, disorder or condition the treatment of which can be due to or associated with the target molecule. Diagnostic methods of such disease, disorder or condition are well known by the man skilled in the art.

In a particular embodiment, the disease, disorder or condition presents a resistance to known treatments.

In a particular embodiment, the subject has never received any treatment prior to the administration of the engineered bacterial strain according to the invention.

In a particular embodiment, the subject has already received at least one line of treatment, preferably several lines of treatment, prior to the administration of the engineered bacterial strain according to the invention.

Preferably, the treatment is administered regularly, preferably between every day and every month, more preferably between every day and every two weeks, more preferably between every day and every week, even more preferably the treatment is administered every day. In a particular embodiment, the treatment is administered several times a day, preferably 2 or 3 times a day, even more preferably 3 times a day.

The duration of treatment according to the invention is preferably comprised between 1 day and 20 weeks, more preferably between 1 day and 10 weeks, still more preferably between 1 day and 4 weeks, even more preferably between 1 day and 2 weeks. In a particular embodiment, the duration of the treatment is about 1 week. Alternatively, the treatment may last as long as the disorder and/or disease persists.

In a particular embodiment, the duration of treatment according to the invention is comprised between 2 days and 20 weeks and involves (i) several successive administrations of the engineered bacterial strain and of the milk oligosaccharide, or (ii) a single administration of the engineered bacterial strain and several successive administrations of the milk oligosaccharide, or (iii) several successive administrations of the engineered bacterial strain and several successive administrations of the milk oligosaccharide, the administrations of the milk oligosaccharide being continued at least once after stopping administrations of the engineered bacterial strain.

As used herein, the expressions “duration of treatment” and “administration period” are used indifferently and refer to the period over which the engineered bacterial strain of the invention and/or the milk oligosaccharide are administered to the subject or provided to the environment.

The form of the pharmaceutical or veterinary compositions comprising the engineered bacterial strain of the invention and/or the milk oligosaccharide, the route of administration and the dose of administration thereof can be adjusted by the man skilled in the art according to the type and severity of the disease or disorder (e.g. depending on the target molecule involved in the disease or disorder and its localization in the patient's or subject's body), and to the patient or subject, in particular its age, weight, sex, and general physical condition.

Particularly, the amount of engineered bacterial strains according to the invention and/or the amount of milk oligosaccharide, to be administered has to be determined by standard procedure well known by those of ordinary skills in the art. Physiological data of the patient or subject (e.g. age, size, and weight) and the routes of administration have to be taken into account to determine the appropriate dosage, so as a therapeutically effective amount will be administered to the patient or subject.

For example, the total amount of engineered bacterial strain, for each administration may be comprised between 1 billion and 100 billion cfu of engineered bacteria, from 5 billion to 50 billion cfu, or from 10 billion to 25 billion cfu of engineered bacteria.

For example, the total amount of milk oligosaccharide, for each administration, may be comprised between 4.5 and 18 g of milk oligosaccharide per day, in particular between 9 and 18 g of milk oligosaccharide per day.

Unless defined otherwise, all technical and scientific terms used herein have the same meaning as commonly understood by one of ordinary skill in the art to which this invention belongs.

All publications mentioned herein are incorporated herein by reference. It is understood that the present disclosure supersedes any disclosure of an incorporated publication to the extent there is a contradiction.

It must be noted that as used herein, the singular forms “a”, “an” and “the” include plural referents unless the context clearly dictates otherwise. Thus, for example, reference to “a cell” includes a plurality of such cells (e.g., a population of such cells). Similarly, reference to “a nucleic acid” includes one or more of such nucleic acids.

### BRIEF DESCRIPTION OF THE SEQUENCES

| SEQ ID NO: | Organism | Description | Type |
|---|---|---|---|
| 1 | Bifidobacterium longum | Blon_2171 | protein |
| | subsp. infantis | | |
| 2 | Bifidobacterium longum | Blon_2173 | protein |
| | subsp. infantis | | |
| 3 | Bifidobacterium longum | Blon_2174 | protein |
| | subsp. infantis | | |
| 4 | Bifidobacterium longum | Blon_2175 | protein |
| | subsp. infantis | | |
| 5 | Bifidobacterium longum | Blon_2176 | protein |
| | subsp. infantis | | |
| 6 | Bifidobacterium longum | Blon_2177 | protein |
| | subsp. infantis | | |
| 7 | Bifidobacterium longum | galactose-1-phosphate | protein |
| | subsp. infantis | uridylyltransferase | |

Example: Treatment of Phenylketonuria that Leverages the Coupling of HMO Internalization Capability to Heterologous Phenylalanine Ammonia Lyase and L-Amino Acid Deaminase Genes Expression

Phenylketonuria (PKU) is a rare disease caused by biallelic mutations in the PAH gene of patients cells that result in an inability to convert phenylalanine (Phe) to tyrosine, elevated blood Phe levels and severe neurological complications if untreated. Most patients are unable to adhere to the protein-restricted diet, and thus have a high Phe level in the gut and do not achieve target blood Phe levels.

A Bifidobacterium longum subsp. infantis strain is genetically engineered to comprise the genes encoding phenylalanine ammonia lyase and L-amino acid deaminase, which allow for bacterial consumption of Phe within the gastrointestinal tract, and therefore reduction of blood Phe level in the subject. The engineered B. infantis strain originally comprises the 2′FL transporter gene that confers a competitive advantage to this strain among the already engrafted Bifidobacteria once administered orally to the patient together with 2′FL.

Natural evolution is a destructive force for synthetic biology, a force that can act counter to engineered constructs in an attempt to rid the organism of any costly unessential processes. To increase the chances that the genes encoding functional phenylalanine ammonia lyase and L-amino acid deaminase stably remains in the engineered B. infantis strain, the expression of these genes which drive unessential processes for the bacteria itself is directly coupled to the expression of the 2′FL transporter gene which on the contrary provides a strong competitive advantage to the bacteria. To achieve this, the three genes are engineered so as to be part of a single operon, with the 2′FL transporter gene placed as the last gene of the operon.

This system ensures that bacteria in which a frameshift mutation and/or a point mutation leading to a STOP codon takes place in the transgene, would lose their competitive advantage among not only the subject's natural microbiome but also among the rest of the other engineered B. infantis strains administered to the patients in which functional genes encoding phenylalanine ammonia lyase and L-amino acid deaminase remain.

1. A method to modulate the level of or to modify a target molecule in a subject or an environment, said method comprising:
administering in said subject or providing to said environment an engineered bacterial strain comprising
(i) a heterologous or engineered nucleic acid involved in the expression of a molecule of interest, wherein the expression of said molecule of interest directly or indirectly modulates the level of or modifies the target molecule in said subject or environment and
(ii) an autologous gene or gene set involved in the import and/or metabolism of a milk oligosaccharide; and
further administering to said subject or providing to said environment said milk oligosaccharide;
whereby the level of the target molecule in said subject or environment is modulated or the target molecule is modified in said subject or environment.

2. The method according to claim 1, wherein said engineered bacterial is present in the microbiome of said subject or environment at a colonization level enabling an overall production of the molecule of interest in an amount efficient for modulating the level of or modifying the target molecule at a rate leading to an effect on said subject or said environment, or on said subject's or environment's microbiome.

3. The method according to claim 1, wherein said engineered bacterial strain is permanently present.

4. The method according to claim 1, wherein said engineered bacterial strain is temporarily present.

5. The method according to claim 1, wherein said engineered bacterial strain becomes present at a colonization level corresponding to at least 5% of the microbiome of the subject.

6. The method according to claim 1, which is for reducing the level of a target molecule.

7. The method according to claim 6, wherein said molecule of interest is involved in the degradation, inactivation, adsorption, absorption, and/or transport of said target molecule.

8. The method according to claim 6, wherein said method is for preventing or treating, in said subject, a disease, disorder or condition associated with said target molecule.

9. The method according to claim 1, which is for increasing the level of a target molecule.

10. The method according to claim 9, wherein said molecule of interest is involved in the expression, secretion and/or activation of said target molecule, or said molecule of interest is said target molecule.

11. The method according to claim 9, wherein said method is for preventing or treating, in said subject, a disease, disorder or condition, a therapy of which comprises said molecule of interest.

12. The method according to claim 11, wherein said engineered bacterial strain becomes present at a colonization level enabling an overall production of the molecule of interest at a therapeutically or prophylactically efficient amount.

13. The method according to claim 1, wherein said milk oligosaccharide is a human milk oligosaccharide.

14. The method according to claim 1, wherein said milk oligosaccharide is selected from the group consisting of fucosyllactose, lacto-N-fucopentose, lactodifucotetrose, sialyllactose, disialyllactone-N-tetrose, 2′-fucosyllactose, 3′-sialyllactosamine, 3′-fucosyllactose, 3′-sialyl-3-fucosyllactose, 3′-sialyllactose, 6′-sialyllactosamine, 6′-sialyllactose, difucosyllactoase, lacto-N-fucosylpentose I, lacto-N-fucosylpentose II, lacto-N-fucosylpentose III, lacto-N-fucosylpentose V, sialyllacto-N-tetraose, their derivatives and combinations thereof.

15. The method according to claim 1, wherein said engineered bacterial strain comprises at least one gene of the H5 gene cluster from Bifidobacterium longum subsp. infantis.

16. The method according to claim 1, wherein said engineered bacterial strain is a Bifidobacterium strain.

17. The method according to claim 16, wherein said engineered bacterial strain is from a subspecies which is not a resident Bifidobacterium subspecies of a typical adult microbiome.

18. The method according to claim 17, wherein said engineered bacterial strain is a Bifidobacterium longum subsp. infantis strain.

19. The method according to claim 1, wherein the expression of said heterologous or engineered nucleic acid is regulated by said milk oligosaccharide.

20. The method according to claim 19, wherein said heterologous or engineered nucleic acid is operably linked to a promoter inducible by the presence of said milk oligosaccharide.

21. The method according to claim 20, wherein said inducible promoter is not the natural promoter of said heterologous or engineered nucleic acid.

22. An engineered bacterial strain comprising (i) a heterologous or engineered nucleic acid involved in the expression of a molecule of interest, and (ii) an autologous gene or gene set involved in the import and/or metabolism of a milk oligosaccharide.

23. The engineered bacterial strain according to claim 22, wherein said milk oligosaccharide is a human milk oligosaccharide.

24. The engineered bacterial strain according to claim 22, wherein said milk oligosaccharide is selected from the group consisting of fucosyllactose, lacto-N-fucopentose, lactodifucotetrose, sialyllactose, disialyllactone-N-tetrose, 2′-fucosyllactose, 3′-sialyllactosamine, 3′-fucosyllactose, 3′-sialyl-3-fucosyllactose, 3′-sialyllactose, 6′-sialyllactosamine, 6′-sialyllactose, difucosyllactoase, lacto-N-fucosylpentose I, lacto-N-fucosylpentose II, lacto-N-fucosylpentose III, lacto-N-fucosylpentose V, sialyllacto-N-tetraose, their derivatives and combinations thereof.

25. The engineered bacterial strain according to claim 22, wherein said engineered bacterial strain is an engineered Bifidobacterium strain.

26. The engineered bacterial strain according to claim 25, wherein said engineered bacterial strain comprises at least one gene of the H5 gene cluster from Bifidobacterium longum subsp. infantis.

27. The engineered bacterial strain according to claim 25, wherein said engineered bacterial strain is from a subspecies which is not a resident Bifidobacterium subspecies of a typical adult microbiome.

28. The engineered bacterial strain according to claim 25, wherein said engineered bacterial strain is a Bifidobacterium longum subsp. infantis strain.

29. The engineered bacterial strain according to claim 22, wherein the expression of said heterologous or engineered nucleic acid is regulated by said milk oligosaccharide.

30. The engineered bacterial strain according to claim 29, wherein said heterologous or engineered nucleic acid is operably linked to a promoter inducible by the presence of said milk oligosaccharide.
